(19) United States
(12) Patent Application Publication
(10) Pub. No.: US 2011/0318375 A1
(43) Pub. Date: Dec. 29, 2011

(54) Immunomodulatory Therapeutic Agents

(76) Inventors: Mitchell J. Melling, Beaver, UT (US); Wade M. Melling, Beaver, UT (US)

(21) Appl. No.: 13/203,045

(22) PCT Filed: Mar. 9, 2010
(86) PCT No.: PCT/US10/26665

§ 371 Date: Sep. 13, 2011

Related U.S. Application Data
(60) Provisional application No. 61/158,526, filed on Mar. 9, 2009.

(51) Int. Cl. A61K 39/00; A61P 37/06; A61K 38/36; C07K 7/06; C07K 7/08

(52) U.S. Cl. 424/185.1; 530/329; 530/328; 530/327; 530/326; 514/13.6

(57) ABSTRACT

A group of peptides has been isolated from the serum of domesticated mammals and then identified through the use of mass spectrometry. These peptides are byproducts of fibrinogen activation and the complement cascade. The peptides of greatest activity are the activated forms of fibrinopeptide A and fibrinopeptide B {activated by the removal of the terminal Arginine), and an immunomodulatory fragment of Complement Component 3. These form of fibrinopeptides A and B have remarkable immunomodulatory ability, enhancing recognition of foreign substances including infectious agents of all types, decreasing the inflammatory response, preventing the deposition of extravascular fibrin, stimulating the resorption of fibrin that has already been deposited, enhancing the body's ability to recognize and eliminate neoplastic cells, decreasing symptoms of allergic reaction including allergic rhinitis and anaphylaxis, decrease the formation and deposit of autoantibody complexes, ameliorate the symptoms of chronic neurologic disease, and decrease the symptoms of chronic pain syndromes.

### REFERENCE TO RELATED APPLICATIONS

This application claims priority to U.S. Provisional Application No. 61/158,526 entitled “Therapeutic Activity of Immunomodulatory Agents” filed Mar. 9, 2009, the entirety of which is hereby incorporated by reference.

### RIGHTS IN THE INVENTION

This invention was made with support from the United States government, from the National Institutes of Health Contract No. NO1 15435, which was awarded by the National Institute of Allergy and Infectious Diseases of the National Institutes of Health, and the United States Government has rights in this invention

### BACKGROUND

1. Field of the Invention

This invention is directed to components isolated from biologic fluids (blood, serum, or exudates), to methods for isolating such components from natural sources, to methods for utilizing these components to maintain and enhance normal function of the human body, and to method for treating diseases and disorders comprising administration of therapeutically effective amounts of the isolated components of either natural or synthesized forms.

2. Description of the Background

In healthy multi-cellular organisms, homeostasis is impeccably maintained through a tightly regulated balance of up and down regulation of various cellular functions. The loss of this homeostatic balance at the cellular level results in chronic disease. These cellular regulatory activities also result in interaction between cell and interaction between systems. These interactions result in an intricate relationship between cells within each of the body's organs as well as interactions between each of these organs/systems. This interaction takes place by several forms of communication such as cytokines, enzymes, and circulating cells.

Any significant insult to the body causes the release of a group of peptides which have profound immunomodulatory effects. These peptides may fall into the broadly described category of cytokines (nonantibody proteins released as a response to a specific stimulus and which act as intercellular mediators), and they may act as enzymes. In either case, they trigger a pathway which slowly moves the body toward a response with one or more of the following characteristics:
1) A rapid decrease of pain and swelling with a persistent analgesic response;
2) A stimulation of the adaptive immune response;
3) Delayed stimulation of the breakdown and resorption of Fibrin deposits;
4) Stimulation of surveillance cells of the immune system (NK cells, T Killer Lymphocytes); or
5) A shift of the baseline status of the organism to an anti-inflammatory state.

While many cytokines have been identified and their roles in specific responses partially described, a limited knowledge of this aspect of cellular regulation is yet available. This is a very active area of research as demonstrated by the contradictory established activity for many of the cytokines. Fully unraveling the intricate intercellular communication carried out through cytokines will necessitate years of additional research.

The integumentary system comprises the single greatest defense against pathogens in all higher forms of life. At the time when an organism is most vulnerable to exposure to a pathologic insult due to an interruption of the integumentary system, the organism utilizes this immune system up-regulation to protect itself from any potential infectious processes. In the cytokine/immune cell cascade, the type of modification which occurs through these peptides enhances the ability of the organism to recognize and respond to the plethora of pathologic insults likely to occur when the skin is breached, while strongly suppressing the inflammation this type of stimulation usually causes. This includes the activation of immune system cells and the release of cytokines which as a therapeutic can enhance the organism's ability to recognize and respond to other pathologic insults, including acute and chronic bacterial infections, acute and chronic viral infections, parasitic diseases, and even neoplastic processes. While skin represents the most important barrier to infection, the innate immune system enables a rapid response to the myriad of attacks resulting from a breach in the integument. The innate immune system recognizes and eradicates pathogens and harmful foreign molecules and has a role in the surveillance and rejection of tumors (Auf et al., 2001; Bacha et al, 2004; Gorelik et al., 1995; Wu and Pruett, 1999).

Besides stimulating the innate immune system, this response also facilitates wound and tissue healing through resorption of fibrin. These deposits are exacerbating factors in many chronic diseases, resulting in the deactivation of many of the healing mechanisms and blocking the nutritional support of the damaged cells. This effect is seen in chronic wounds as well as the plaques of multiple sclerosis, the neurofibrillary tangles of Alzheimer's disease, the plaques of Atherosclerosis, the tissue changes of autoimmune diseases, and the fibrin deposits around cancer cells which act as a shield protecting the tumor from the immune system.

This response also facilitates wound healing and immunomodulation decreases the effects of inappropriate antibody expression by stimulating the production of T-killer lymphocytes. These cells seek out and destroy B-Cells which are inappropriately producing auto-antibodies. By eliminating the production of auto-antibodies, the attack on the body is stopped and the inflammation these antibodies produce is eliminated, decreasing the symptoms and often the most significant cause of autoimmune disease. These peptides also enhance the ability of an organism to recognize and destroy cells manifesting abnormal protein on their cell wall. As all cancer cells express abnormal cells on their cell membrane, these T-killer lymphocytes are essential in the recognition and destruction of all types of cancer.

In addition to this increased surveillance, these peptides have a strong anti-inflammatory activity. Through stimulation of TH-2 cytokines, these peptides suppress the inflammatory response that otherwise would be expected in acute injury. In addition, they appear to be even more helpful in blocking inflammation from chronic processes. This anti-inflammatory effect enhances the healing of both acute and chronic injuries.

In chronic disease processes, the deposition of fibrin in the extra-vascular space is an integral part of the progression of many diseases. Mobilizing these chronic fibrin deposits and preventing their deposition have now become targets for therapeutics, but no successful therapeutics which work through these mechanisms have previously been identified. Preventing fibrin deposits from occurring in response to an acute pathologic process prevents the transition of these acute processes into chronic disease.

Fibrinogen is a bivalent protein composed of six polypeptide chains, two each of the Aα, Bβ and γ chains. These chains are linked together near their Amino termini by disulfide bonds, leaving their carboxy termini exposed to the action of thrombin. In response to injury to a blood vessel wall, thrombin is activated and cleaves fibrinogen to produces a fibrin monomer and two peptides each of fibrinopeptides A and B. These fibrin monomers then bind to each other to form a loose scaffolding for clot formation. The released fibrinopeptides have been characterized, with some species dependant activities, but they have previously not been identified as modulators of the immune system. They also have the therapeutic benefits of acutely decreasing vascular permeability and triggering a delayed stimulation of the resorption of fibrin from the intimal and extra vascular spaces, benefits which have not been previously identified or recognized. Throughout the systems of the body, any activity which produces a change in our homeostasis also stimulates an opposite process to allow for the reversal of that change. One therefore would expect a process that causes a blood vessel to leak to also result in a process of correcting that leak. In the case of Fibrin production, this is attained by two mechanisms: 1) the production of a clot to seal the area of leakage, and 2) the release of molecules which decrease the overall vascular permeability to prevent the migration of these molecules into areas where they are not needed and can be harmful. In addition, the presence of fibrin in the extravascular space and subintimal space are harmful on a long term basis but necessary acutely. Fibrinopeptide A produces a delayed resorption of these fibrin deposits to prevent the problems associated with their chronic presence. When this resorption fails, chronic disease result. The cross species activity of these peptides is known, but the mechanism of this interspecies activity has not been previously explained. While Fibrinopeptide B possesses little to no homology from species to species, the terminal sequence of Fibrinopeptide A has significant homology through most mammals, likely accounting for most of this cross species activity. In addition, a portion of C3 bears considerable homology from one mammal to another.

The deposition of fibrin into the intima of blood vessels in coronary artery disease and into the extra-vascular space in many other diseases results in the progression of these diseases. Mounting data on the regulation of fibrin indicates that this fibrin deposition is a major part of many chronic disease processes. This is due not only to the impairment of function caused by the physical barrier fibrin forms, but also the pro-inflammatory activity of fibrin in these spaces. In addition, the presence of fibrin in these spaces suppresses the activity of some cells which are essential for healing. One example of this is the ability of extra-vascular fibrin to inactivate the regenerative activity of Swann Cells. Over the last several years researchers have been able to demonstrate the benefit of removal of extra-vascular fibrin in many of these disease processes. These studies demonstrate the proinflammatory activity of fibrin as well as the impairment of normal cellular/organ function. This impairment is a major component of the pathologic process of many diseases, including but not limited to Multiple Sclerosis, Rheumatoid Arthritis, Peripheral nerve crush injury, Alzheimer's Disease, macular degeneration, chronic wounds and Atherosclerosis. In these studies extra-vascular fibrin was an important target for new therapeutics.

Over the last several years Th1 and Th2 cytokines have also become a prominent focus in the study of the immunologic/inflammatory response. Initially, Th2 cytokines were viewed as anti-inflammatory and Th1 cytokines were viewed as pro-inflammatory. This generalization does not fully describe the complex and intricate interaction between these opposite ends of the inflammatory spectrum. Alternatively, the inflammatory response has also been characterized into active and passive components, and further still, into portions active in the innate and adaptive immune system. None of these divisions truly describes the response seen in vivo, as the systemic response nearly always incorporates a combination of Th1 and Th2 activities, innate and adaptive, and active and passive immunologic/inflammatory responses.

This combination activity also occurs in an organism's response to these peptides. These peptides cause a spectrum of activities in the cytokine cascade that could be viewed as either stimulatory or suppressive, but the net result is a stimulation of the immune system, stimulation of the removal of fibrin from the extra-vascular space, and suppression of inflammation.

Many different proteins and peptides circulating in the bloodstream are well recognized for their effects on the local inflammatory processes in humans as well as in experimental animal models. These proteins and peptides include a variety of cytokines and chemokines. These substances produce a feedback loop that regulates the extent of the local inflammatory response. In most chronic diseases this control over the inflammatory response is inadequate, allowing localized inflammation to result in the destruction of healthy tissues. Fibrinopeptide A has an anti-inflammatory affect in a variety of disease models, thus decreasing local tissue destruction and ameliorating the disease state. These models are herein described, demonstrating the anti-inflammatory effect of Fibrinopeptide A. The mechanism of action of this anti-inflammatory response is demonstrated the specific ability of fibrinopeptide A to produce the shift in the cytokine panel from a predominantly Th1 response to a predominantly Th2 response through the production of specific anti-inflammatory cytokines. In addition to this cytokine shift, fibrinopeptide A has the ability to decrease vascular permeability. This decrease in the vascular permeability has the effect of maintaining the plasma proteins (such as fibrin) within the blood vessels, preventing their pro-inflammatory activity in the extra-vascular space. Although Fibrin in particular has been implicated as a pro-inflammatory molecule in the extra vascular space, Fibrinopeptide A has the ability to greatly reduce the migration of fibrin from the blood stream into the extra-vascular space, and to expedite the removal of fibrin from this space. This anti-inflammatory activity (or at least prevention of a pro-inflammatory activity) has profound implications in the treatment of chronic inflammatory conditions.

In 1978, Ruhenstroth-bauer et. al. demonstrated the anti-inflammatory activity of Fibrinopeptides A and B. In their research, Ruhenstroth-bauer and associates sought an understanding of the specific cause of inflammation in response to a pathologic challenge. They first demonstrated a shift in the acute phase proteins released after a pathologic insult. They then isolated an anti-inflammatory activity of the proteins produced by this shift. U.S. Pat. No. 4,215,109 describes the process of further isolating this anti-inflammatory activity first to fibrinogen, and then further testing confirmed Fibrinopeptides A and B to be the source of this biologic anti-inflammatory response, a response that could have a beneficial effect in almost all pathologic processes. They demonstrated this benefit in a carrageenin-induced rat paw edema model, demonstrating the benefit of increased fibrinogen injected intra-peritoneally, then isolating this beneficial activity to fibrinopeptide A and B. However, their research failed to describe the mechanism of action of this anti-inflammatory activity or even isolate this activity to a specific peptide. They did not utilize their research to produce a therapeutic.

This same group (Scherer et. al, 1981) subsequently demonstrated this anti-inflammatory effect in another disease model. Improvement in the course of Experimental Allergic Encephalomyelitis (EAE) in guinea pigs and rats (a disease model for Multiple Sclerosis (MS)) by daily intraperitoneal injections with human fibrinopeptides A and B was demonstrated. These injections produced significant amelioration of the disease state in the treated animals as compared to controls. Improvements in the clinical neurological signs of the disease were evident in that the number, the severity and the duration of pareses were diminished in treated animals. Furthermore, the inflammatory alterations of vasopermeability associated with extravasation of plasma proteins and edema of the neuroparenchyma were significantly less pronounced in the fibrinopeptide-treated animals. Finally, a significantly higher titre of circulating immune complexes was observed in the serum of these animals, demonstrating that treatment with fibrinopeptides A and B did not alter the specific immune response to the antigenic challenge. They therefore concluded this anti-inflammatory response is not at the expense of immunosuppression. No differences in anti-basic protein and anti-brain antibody production were observed. The characteristic cellular infiltrates of EAE also showed no significant qualitative or quantitative differences between fibrinopeptide-treated animals and the saline-treated controls, (Scherer et. al. 1979) but the inflammation typically identified in conjunction with these findings was much less pronounced.

Shortly after these studies were completed, Marusic et. al. demonstrated similar findings with the induction of any type of peritonitis. As these peptides are mildly acidic, the research by Scherer et. al. was likely viewed as a false positive, and there does not appear to be further published research from anyone into this pathway. Fibrin deposition is a significant contributing factor (Adams et al. 2004) in the development of plaques in MS, and these deposits form as a consequence of the leaky vasculature. As described above, Scherer et. al. demonstrated that this vascular leakage was ameliorated by treatment of Fibrinopeptides A and B.

Decreased vascular permeability also slightly reduces the migration of immune complexes, and slows the deposition of fibrin in the extra-vascular space. In addition, fibrin has the ability to regulate Schwann cell differentiation by maintaining Swann cells in a nonmyelinating state. Fibrin induces phosphorylation of ERK1/2 and production of p75 NGF low affinity receptor in Schwann cells which maintains them in a nonmyelinating state, suppresses fibronectin production, and prevents synthesis of myelin proteins. (Akassoglou, et. al., 2002). In many chronic neurologic diseases this continued presence of Fibrin in the extra-vascular space is implicated in the persistence of the disease process and progression of neurologic symptoms. These include the presence of fibrin in Multiple Sclerosis Plaques, Alzheimer's disease neurofibrillary tangles, Basil Ganglion lesions in Parkinson's disease, Peripheral nerve lesions in Chronic Inflammatory Demyelinating Polyneuropathy, and many others. These fibrin depositions are also an important part of the pathologic process outside of the neurological system. This is demonstrated by the essential role of fibrin in the development of atherosclerotic heart disease, chronic wounds, Hypertension, Cancer (creating a barrier around the cancer cells), macular degeneration, Autoimmune diseases, and many others.

Anti-Allergenic/Anti-Anaphylaxis Activity have also been observed. Masuda et. al. (2001) demonstrated that a Fibrinopeptide A fragment (the same fragment identified as the object of this invention) deglycosylates mouse antibody IgE. This deglycosylated IgE no longer had the ability to stimulate histamine release from Mast Cells, thus preventing an anaphylactic/allergic response. This deglycosylation however did not affect the ability of IgE to interact with the antigen, bind with mast cells and other immunologic cells, or alter the ability of these cells to perform all of their other normal activities resulting from IgE antigen/antibody complex attachment to their membrane receptor. They further demonstrated that the synthetic form of the peptide sequence also deglycosylated IgE in a similar fashion. Once this deglycosylation had occurred, they were no longer able to induce mouse systemic anaphylaxis (Masuda et. al. 2001). However, Masuda and associates failed to elucidate the mechanism of this deglycosylation. They also did not evaluate any further effect the deglycosylated IgE may have on the immunologic response. The modification of the systemic effect of cytokines through glycosylation and deglycosylation is well established. This deglycosylation of IgE may be a direct effect of the Fibrinopeptide A on IgE, or it may be an additional effect of the Immunomodulation. This deglycosylation and subsequent lack of histamine response may partially explain the decreased permeability of blood vessels seen after administration of Fibrinopeptide A. A lack of histamine response may result in a decrease in the fibrin deposition at the sight of insult. As this deglycosylation does not appear to suppress the immune system in any other way and does not even alter the ability of IgE to attach to an antigen, the beneficial effect comes with no detrimental effect on an organism's immunologic response. Rather, this deglycosylation just controls the detrimental acquired hyperactivity which causes allergic reactions.

The ability of Fibrinopeptide A to decrease the severity of injury in a burn model has been demonstrated. (Wormser, Uri patent Ser. No. 10/790,888). In this patent fibrinopeptide A in conjunction with Histone peptides was found to prevent injury from thermal and chemical burns and speed healing of burns that had already occurred. They postulate this healing occurs primarily due to the anti-inflammatory effect. To demonstrate this they pretreated with the exudates from burns of other animals of the same species, and then isolated the fraction of the exudates responsible for this benefit. They found that a fraction containing only Fibrinopeptide A had tremendous protective effect in decreasing the severity of burn. They did not postulate a mechanism of action for this protective effect. This work indicates the mechanism of this effect through the decreased permeability of blood vessels which prevents the damage that the extravasation of the plasma proteins causes, including the release of lysosomal enzymes and the production of superoxide anions.

The ability of fibrinopeptide B (and possibly A) to facilitate wound healing has also been demonstrated by the ability of Fibrinopeptide B to enhance migration of fibroblasts, monocytes and neutrophils into the area of injury, without stimulating the release of lysosomal enzymes or the production of superoxide anion from these neutrophils (Senior. et. al., 1986). The release of these substances in response to the chemo attractant activity of fibrin toward neutrophils is postulated to result in the demyelination seen in Multiple Sclerosis. In addition, Gray et. al. (1990) demonstrated the ability of the fibrinogen alpha and beta chains to stimulate the replication of fibroblasts, and this activity was significantly enhanced by the addition of thrombin to a fibrinogen containing solution, strongly suggesting this activity is associated with the products of this cleavage.

These finding have not been recognized as sufficient to develop a therapeutic containing them, as demonstrated by the lack of information and research continued in this area after the discovery of these peptides decades ago. This lack of ongoing research is at least partially due to the failure of all of these researchers to recognize the immunomodulatory and anti-inflammatory ability of these peptides, enhancing the immune system while decreasing the inflammatory response. This immunomodulation aids in the prevention of chronic infection, promotes a much healthier Th2 environment, and stimulates migration of cells necessary in wound healing, while preventing the mechanisms of damage which slow healing and lead to chronic wounds.

The growth of new blood vessels is a complicated multifactorial process involving cells of several different types. When vascular injury interrupts the flow of blood, the healing process necessitates restoration of blood flow to the healing cells. This process begins with the degradation and absorption of injured cells and thrombus coupled with migration of fibroblasts to fill the injury defect. Then vascular cells differentiate to form tubules which eventually mature into blood vessels. Angiogenesis is essential to the normal physiological processes of wound healing.

Fibrin accumulates around leaky blood vessels in solid tumors (Brown, et. al. 1988). Fibrin has also been shown to polymerize at the host-tumor interface to form fibrin networks that can promote tumor angiogenesis by supporting the adhesion, migration, proliferation and differentiation of endothelial cells (Bootle-Wilbraham et. al. 2001). As this fibrin network thickens this promotion of angiogenesis is lost and the fibrin network becomes a barrier to adhesion, migration, proliferation, and differentiation of these endothelial cells. This barrier also prevents immune cells from recognizing and eliminating tumor cells. By only utilizing the activated fragments fibrinopeptides A and B, the resulting cytokine cascade has these beneficial angiogenic activities without “protecting” tumor cells from immunogenic attack. This effect is partially explained through extrapolation of the available data on Interleukin 1B and the effect it has on inflammatory and immune cells. The migration and differentiation of cells in a wound bed are greatly affected by this cytokine and the presence of certain other immune cells (macrophages and lymphocytes). IL-1 B enhances migration of these cells into the wound bed, thus producing an environment which is more conducive to angiogenesis. In addition, the elevation of IL-10 in response to this activated fragment of Fibrinopeptide A and other direct effects of fibrinopeptide A on the inflammatory cascade offer an enhanced ability of lymphocytes, monocytes, macrophages, and monocytes to migrate into these areas without the release of lysosomal enzymes but still with the ability to attack cells and other foreign bodies.

The deposition of Fibrin in the walls of blood vessels occurs in many disorders of the vascular system. The ability of Fibrinopeptide A to mobilize these fibrin depositions and to prevent further deposition has far reaching implications in all vascular disease. These include, but are in no way limited to, improvements in Coronary Artery Disease, Macular Degeneration, Claudication, and Atherosclerosis. In many additional diseases this process enhances blood flow, improving the outcome in most chronic diseases. This absorption of these fibrin deposits creates an environment which is more conducive to the angiogenic process when an injury to any tissue occurs. This is best exemplified in the diabetic foot ulcer, in which the chronic fibrin deposition in the macro and micro vasculature greatly impedes blood flow and prevent tissues from healing due to the lack of circulation to the affected area.

No previous studies document the ability of Fibrinopeptide A to stimulate angiogenesis and, thus, a healthy vascular environment. In fact, Staton et. al. (US Pat. Application Publication No. 20040039157) demonstrated that one fragment of Fibrinopeptide A produced during cleavage by plasmin actually had a quite prominent anti-angiogenic activity. Thompson et. al (1992) also isolated the angiogenic activity of fibrinogen to fragment E, a portion of fibrinogen at the central knot released by plasmin, but failed to recognize the potential therapeutic effect of Fibrinopeptide A, a byproduct which should have been present in the solution he demonstrated to have the therapeutic activity, as Thrombin was initially used in the solution to breakdown Fibrinogen.

No published studies establish Fibrinopeptide A and/or B as an antiviral or antibiotic. An increased survivability of mice treated with Fibrinopeptide A and then given Ponto Toro Virus was observed. Two different forms of Fibrinopeptide A were utilized: 1) a filtered serum fraction of goat serum calculated to contain approximately 3 mg of Fibrinopeptide A (also containing goat Fibrinopeptide B and a fragment of Complement C3), and 2) synthetic Fibrinopeptide A. While these substances did not perform as well as a direct anti-viral (an expected outcome), the results did demonstrate improved survival of the treated animals when compared to the placebo group. (See FIG. 1). In this study several criteria were analyzed. These included Liver, spleen, and serum virus titers; Serum alanine aminotransferase (ALT) determinations; livers and lungs were scores for hepatic icterus on day 3 of infection; daily weight measurement; Mean Day to Death; and overall survivability. The two groups treated with test articles containing fibrinopeptide A performed identically. In these treatment groups 60% of the mice lived, while in the placebo group only 25% survived. This improvement was statistically significant for each of the fibrinopeptide A groups independently (P value=0.03), and when these groups are combined to calculate the overall improvement with Fibrinopeptide A the statistical significance improved (P value=0.015). This increased survivability occurred even though there was no observable difference in any of the other disease criteria evaluated, indicating no change in the ability of the virus to cause disease, but rather an increased ability of the organism to fight off a life threatening infection following one dose of Fibrinopeptide A. No measure of inflammation or fibrin deposition was performed in this study. A difference exists between Fibrinopeptide A and the Ribavirin control, this difference was not statistically significant (P value=0.08).

These results demonstrate the potential therapeutic value of Fibrinopeptide A in the infectious disease arena, with the ability to augment healing and decrease the duration of symptoms. As the body's normal response to infectious diseases results in a very pro-inflammatory state, even after the infection is cured this state often causes persistent symptoms. Fibrinopeptide A has the ability to alleviate these symptoms and to therefore shorten the symptomatic phase of the disease without blocking the body's ability to fight off the infection. This effect also may be due to the ability of Fibrinopeptide A to mobilize proteins out of the extra-vascular space.

Given the delay in the shift of the cytokine panel and based on the anticipated effects of this demonstrable shift, the enhanced immunity produced by fibrinopeptide A has a much stronger effect on the adaptive immune response than on the innate immune response. This difference accounts for lack of improvement in all of the other measures of disease while still greatly enhancing survivability.

A second study was conducted to test these substances against Influenza A H1N1. In this study the control was low dose Ribavirin. All of the mice died in every group, suggesting a more severe infection than anticipated. This again demonstrates the lack of time to develop a true adaptive response which would have been enhanced by the presence of Fibrinopeptide A.

The anti-neoplastic activity of the peptides of this invention in treating neoplastic disease are due to three different mechanisms of action: 1) increased surveillance by the immune system to eliminate neoplastic cells, 2) preventing or eliminating the deposition of fibrin around cancer cells, and 3) decreasing the swelling around tumor cell clusters and the symptoms this swelling causes.

Primarily through the activity of IL-1B the immune system production of T-killer lymphocytes, NK lymphocytes, and B cells is increased. This differentiation allows the organism's immune system to seek out and destroy cancer cells based on the abnormal proteins manifest on their cell membranes. These peptides therefore have the ability to treat even those cancers that respond poorly to chemotherapy. While this mechanism of action will require time to attack and remove cancers that have already spread, this type of stimulation can also prevent cancer from ever developing.

Increased plasma fibrinogen levels or secretion of fibrinogen by the malignant tumor cells themselves cause the deposition of fibrinogen or fibrin into the extracellular matrix of the malignant tumor tissues, and these factors have the effect as part of the extracellular matrix to promote proliferation, invasion and metastasis of the malignant tumor cells (Rybarczyk et. al. 2000). The ability of these peptides to prevent the migration of fibrin into the matrix surrounding the tumor cells will therefore have the effect of eliminating this protection of cancer cells from the host's immune system and facilitate recognition and elimination of cancer cells by the host. In addition to this effect, the stimulation of the host immune system by these peptides enhances the ability of the immune system to destroy these cells.

The anti-inflammatory activity described above decreases the symptoms of metastatic cancer, as many of these symptoms are due to the inflammation the metastasis cause. In addition, the symptoms commonly caused by chemotherapy are partially due to the inflammatory effect of these medications and the cellular destruction these medications cause.

From the above description, these peptides have the ability to treat auto-immune disease by: 1) decreasing the inflammatory response to an auto-immune antibody attack, 2) decreasing the fibrin depositions which lead to the progression of auto-immune disease pathology, and 3) destroying the B-cells which produce auto-antibodies through the production of T-killer lymphocytes which seek out and destroy cells producing auto-antibodies. The loss of the ability to perform this surveillance function is ultimately responsible for the development of autoimmune disease. These peptides have the ability to restore this function. While IL-1β has been implicated in progression of the destructive process of some diseases, this low level stimulation does not seem to have these effects or the presence of IL-10 stimulation mediates/prevents these effects.

Buckheit (WO/2006/116381) demonstrated that a serum fraction from goats treated with cancer cell lysates has an anti-neoplastic activity toward that particular cancer. While this was initially thought to be secondary to antibody formation in goats, they subsequently demonstrated that the serum fraction from these animals depleted of the large proteins (including immunoglobulin) still contain this anti-neoplastic activity. They also demonstrated the ability of a serum fraction from a goat pretreated with cancer cell lysates from one type of cancer to treat a different type of cancer. They postulate that this effect is related to antibody fragments.

Immunization or vaccination involves exposing a patient to inactivated pathogenic antigens in order to stimulate an immune response to that specific pathogen. This active type of immunity typically provides long term protection against that specific disease. Extensive attempts to establish active immunity toward several common viruses have proven futile to date, and this has led to research into the utilization of passive immunity to treat these diseases. This type of therapy utilizes neutralizing antibodies produced by one or many patients or animals to treat infection in another patient or animal. Historically, passive immunity has been utilized to treat a variety of diseases. For many decades, immuno-compromised patients have been given pooled IgG to enhance their immunity. With the increase incidence of blood born infection in our population and the ability to produce monoclonal antibodies, this therapy has fallen out of favor for the treatment of general mild immune system dysfunction. Pooled antibody preparations are only rarely used now to boost the immune system in times of increased exposure, and to stop the attack of autoimmune disease.

Despite these factors, passive immunity has continued to receive attention as a possible therapeutic for certain viral and bacterial infections. However, the serum from individuals or animals with established immunity might also contain the virus or bacteria, thus, transfer of serum could result in an infection as well. In an effort to develop new anti-viral and antibiotic drugs to specific diseases, this hyper-immune serum has been evaluated for therapeutic potential to humans afflicted with these diseases. These approaches carry the obvious difficulties of the occurrence of hypersensitivity reactions and the potential for additional infection, but they have demonstrable efficacy. The most simplistic form of this type of therapy is performed by simply exposing a host animal to a particular pathogen and then extracting blood from the animal and injecting the serum fraction containing the antibodies into the patient.

Karpas (U.S. Pat. No. 4,863,730) utilized a preparation containing a high titer of heterologous human neutralizing antibodies obtained from the plasma of HIV positive patients to treat HIV. While this method proved beneficial in decreasing viremia and delaying onset of AIDS, clinical application and large scale production are exceptionally problematic.

Davis (WO 97/02839, WO 01/60156, 02/07760, and US 2002/006022) utilized a method involving inoculation of goats with viral lysates (HIV) or bacterial lysates (Staphylococcus, Steptococcus, E. coli) and then injecting the serum obtained from these hyper-immune goats to treat HIV infected patients. His method and success utilizing this method to treat HIV and other infections have been widely publicized (Washington Post, Apr. 9, 2000; Dateline Houston television broadcast, Sep. 18, 1998; etc.). In his process he utilizes standard extraction and purification methods including ammonium sulfate precipitation followed by a filtration process (dialysis or gel filtration) after allowing the blood to initially clot.

Gelder and associates (U.S. Pat. Nos. 6,043,347, 6,258,599, 6,335,017, and 6,670,181) also developed a method utilizing hyper-immune goats to produce neutralizing antibodies which are hypothesized to recognize certain viral epitopes. They utilize antigens that fail to trigger the production of neutralizing antibodies in humans but are handled appropriately by goats. Gelder complicated the process described by Davis by injecting purifying proteins from HIV-1 MN and HIV-2 NZ into goats, and then augmenting the immunity with synthetic peptides from regions known to contain highly conserved HIV epitopes. This method has lead to production of a medication (HRG214) which is currently in clinical trials for the treatment of HIV. The manufacturer also claims that its serum prepared from animals exposed to one virus through their process is beneficial in the treatment of other types of viral diseases. (See Vironyx web site). In addition, depleting the serum of large proteins (including removal of all full antibodies) does not eliminate the benefit but does enhance the safety of the preparation. It is postulated that this benefit is derived from the presence in the remaining serum fraction of antibody fragments (particularly the F_{ab} fragment). In the information accompanying this research, it is stated that it is best to remove all proteins greater than 30 kD in size, essentially eliminating all of the antibodies and fragments that result from the treatment of the goats.

Dalgleish (WO 03/004 049, WO 03/064472) recognized that the activity of some of these formulas could not be fully explained by the activity of neutralizing antibodies. He therefore postulated that the anti-inflammatory activity of these preparations may be dependent upon anti-HLA and/or anti-FAS antibodies. He demonstrated that these anti-bodies have an anti-inflammatory effect, preventing an over-stimulation of the immune system by viral epitopes resembling normal human HLA. Dalglish and associates demonstrated that the serum fraction enriched with these anti-HLA and/or anti-FAS antibodies are useful in the treatment of a wide variety of diseases with inappropriately high HLA levels such as chronic infections (both viral and bacterial), tropical cancers (lung, pancreas, liver, bowel, lymph nodes and skin cancers are specified), and other diseases with high HLA levels such as Diabetes and Multiple Sclerosis. In his research, Dalglish and associates did not utilize hyper-immune goats (no treatment of the goats with antigen prior to removal of the blood).

Tolett (WO 04/033665), also describes the therapeutic benefit of a heterologous serum mixture for treatment of HIV using the filtered, but otherwise unpurified, serum or plasma of HIV-exposed animals. The serum or plasma mixture is simply an unprocessed mixture of serum from various animals that has not undergone any purification process.

Ansley (U.S. Pat. No. 5,219,578) uses a similar preparation process to prepare an IgG serum fraction, although in this patent, no prior stimulation of the goat's immune system is undertaken. The serum of these pathogen naïve goats was removed and processed, and then utilizes to prevent and treat a variety of veterinary diseases. These diseases include equine lower respiratory disease (ELRD) caused by a variety of opportunistic organisms, ovine foot rot in sheep and lambs caused by various serotypes of B. nodosus, and bovine respiratory disease. Ansley demonstrated that the non-immunized goat serum induces non-specific activation of the immune system in the treated animal, resulting in a remarkable therapeutic effect.

Hamm et. al. demonstrated the ability of a caprine serum fraction to treat equine lower respiratory infection.

Thacker (U.S. Pat. No. 7,358,044) demonstrated that a serum fraction containing low molecular weight peptides could be used to stimulate the immune system, greatly improving the survival rate in animals lethally challenged with a variety of pathogens. In this studies, serum from pathogen naïve animals was used in the preparation of the medication. This patent also references studies in which a fraction of caprine serum, substantially free of immunoglobulins, could confer significant protection to chickens challenged with a lethal dose of Pasteurella multocida when the caprine serum fraction was administered 24 hours prior to the bacterial challenge. Similar results were found in mice given a lethal challenged with Salmonella typhimurium.

Buckheit (U.S. Pat. App 2006/0292162) demonstrated the serum or plasma from animals inoculated with lysates from viruses, bacteria, or cancers cells has the ability to treat the disease from which the lysates were prepared. This therapeutic effect is greatest in the serum fraction which is essentially free of all antibodies and large proteins.

In addition to these studies demonstrating the benefit of serum fractions, several studies have been completed exploring the use of neutralizing monoclonal antibodies. The results of these studies have proven disappointing. (see, e.g., Burton D R et al. Science (1994) 266: 1024-1027; Trkola A. et al. J. Virol. (1996) 70: 1100-1108; Conley A J. et al. Proc. Natl. Acad. Sci. USA (1994) 91: 3348-3352;). Although these antibodies seemed to have a significant benefit in vitro, no clear benefit could be demonstrated in vivo (Stiehm, 1995). In general, heterologous antibody mixtures (produced from raw serum and therefore containing the active peptides of this invention) seem to be markedly more beneficial than monoclonal antibodies, again suggesting an alternative mechanism of action to the antibodies alone. These mixtures are also felt to be more beneficial in the prevention of disease than the treatment of disease (Montefiori, 2001).

### SUMMARY OF THE INVENTION

As embodied and broadly described herein, the present invention is directed to pharmaceutical compositions, dietary supplements of these composition, and method for the preparation of a biologically active fraction of mammalian serum from animal blood and isolated and manufactured peptides therefrom to modulate the immune system and enhance the immune response under a variety of conditions. In addition, the invention includes the synthetic forms of these peptides, and the invention includes and derivations and modifications of these peptides that enhance these therapeutic and prophylactic benefits.

One embodiment of the invention is directed to an agent comprising a peptide containing a sequence identified in SEQ ID NOs. 1-5, 7-9, 11-13, 15, 16, or 20-22, a sequence of Fibrinopeptide A, a sequence of a region of Fibrinopeptide A that is substantially homologous between species of mammals that produce Fibrinopeptide A, a sequence of Compliment C3, or any of the foregoing sequences also containing one or more conservative amino acid substitutions, wherein the agent contains substantially no detectable Fibrinopeptide B. Preferably the agent further comprising a pharmaceutically acceptable carrier such as, for example, water, oil, edible oil, fatty acids, lipids, polysaccharides, cellulose, glycerin, glycol, and combinations thereof. A preferable edible oil includes, for example, lemon oil, peppermint oil, or grape seed oil. Preferred agents are formulated for oral, transmucosal, parenteral, lymphatic, or intravenous administration such that the biologically active form of the agent is released into a system of a patient at a physiologically effective concentration. Also preferred is an agent which is a dietary supplement and agents which are purified from biological sources or synthetically manufactured.

Another embodiment of the invention is directed to a pharmaceutical composition comprising Fibrinopeptide A or a fragment thereof, and a pharmaceutically acceptable carrier, wherein the Fibrinopeptide A or fragment thereof is at a therapeutically effective amount. Preferably the therapeutically effective amount is from 0.1 mg to 500 mg. Also preferred is the composition wherein the therapeutically effective concentration prevents deposition and stimulates resorption of fibrin within the extravascular spaces, such as is associated with coronary artery disease, and subintimal spaces in a patient. Preferably the composition is nontoxic at the therapeutically effective concentration and substantially free of detectable Fibrinopeptide B. The composition may caontain Fibrinopeptide A or fragment thereof that are derived from a human or non-human, but preferably mammalian sequence of Fibrinopeptide A. Mammals that express the non-human sequence of Fibrinopeptide A include an equine, a feline, a canine, a bovine, a caprine, an ovine, and a murine.

Another embodiment of the invention is directed to a method for treating or preventing a disorder of a patient comprising: providing a pharmaceutical composition comprising Fibrinopeptide A or a fragment thereof, and not Fibrinopeptide B, and a pharmaceutically acceptable carrier, wherein the Fibrinopeptide A or fragment thereof, is derived from a mammal that is not a human; and administering a dose of the composition to the patient, wherein administration is transmucosal such that the Fibrinopeptide A or fragment thereof achieves a therapeutically effective level within the lymphatic system of the patient within 5 minutes of administration. Preferably the patient is a human, and also preferably the disorder is vascular inflammation or coronary artery disease. The preferred single dosage of the composition contains from 0.1 mg to 10 mg of active ingredient, and preferred administration comprises an initial administration and subsequently, both oral and transmucosal, and a continued administration, and the continued administration is not repeated for an interval of at least 7 days. Preferably the Fibrinopeptide A or fragment thereof stimulates the patient's cells to release cytokines IL1β, IL-10, and not IL-1, IL-4 or TNF\dot{α}. Another preferred aspect is for the activity of Fibrinopeptide B of the patient to be suppressed, such as, for example, by the administration of a Fibrinopeptide B binding agent.

Another embodiment of the invention is directed to a method of preventing deposition of fibrin and absorbing fibrin deposited within blood vessels of a patient, comprising: providing a pharmaceutical composition that comprises Fibrinopeptide A or a fragment thereof and a pharmaceutically acceptable carrier; and administering the composition to a patient such that the Fibrinopeptide A or fragment thereof is at a therapeutically effective level is achieved in the lymphatic system of the patient. Preferably the patient is a human and the Fibrinopeptide A or fragment thereof is derived from a mammalian sequence of Fibrinopeptide A that is not a human. Administration of the composition is preferably directly to the lymphatic system by transmucosal administration, and comprises an initial administration and subsequently, a continued administration, and the continued administration is no more than once a week.

Another embodiment of the invention is directed to a fraction of serum of a mammal wherein the fraction contains multiple components, is clarified of particulates, and substantially all components are within a molecular weight range of from about 1,200 Daltons to about 1,700 Daltons. Preferably the mammal is an equine, a feline, a canine, a bovine, a caprine, an ovine, or a murine.

Another embodiment of the invention is directed to an agent comprising a peptide containing a sequence selected from the group consisting of SEQ ID NOs. 6, 10, 14, and 17-19, a sequence of Fibrinopeptide B, and a sequence of a region of Fibrinopeptide B that is substantially homologous between species of mammals that produce Fibrinopeptide B, wherein the agent contains substantially no detectable Fibrinopeptide A. Preferably the agent further comprising a pharmaceutically acceptable carrier such as, for example, water, oil, edible oil, fatty acids, lipids, polysaccharides, cellulose, glycerin, glycol, and combinations thereof. A preferable edible oil includes, for example, lemon oil, peppermint oil, or grape seed oil. Preferred agents are formulated for oral, transmucosal, parenteral, lymphatic, or intravenous administration such that the biologically active form of the agent is released into a system of a patient at a physiologically effective concentration. Also preferred is an agent which is a dietary supplement and agents which are purified from biological sources or synthetically manufactured.

Another embodiment of the invention is directed to a method for treating or preventing a disorder of a patient comprising: providing a pharmaceutical composition comprising Fibrinopeptide B or a fragment thereof, wherein the composition contains substantially no detectable Fibrinopeptide A, and a pharmaceutically acceptable carrier, wherein the Fibrinopeptide B or fragment thereof, is derived from a mammal that is not a human; and administering a dose of the composition to the patient, wherein administration is transmucosal such that the Fibrinopeptide B or fragment thereof achieves a therapeutically effective level within the lymphatic system of the patient within 5 minutes of administration. Preferably the patient is a human, and the disorder is an auto-immune disorder, such as, for example, arthritis, Crohn's disease, Coeliac disease, diabetes mellitus type 1, Grave's disease, idiopathic thrombocytopenic purpura, psoriasis, scleroderma, systemic lupus erythematosus, or ulcerative colitis, or the disorder is a immunoregulatory disorder, such as, for example, an overactive immune system. The preferred single dose contains from 0.1 mg to 10 mg of active ingredient.

Another embodiment of the invention is directed to a fraction of serum of a mammal wherein the fraction contains multiple components, is clarified of particulates, and substantially all components are within a molecular weight range of from about 800 Daltons to about 2,300 Daltons. Preferably, the mammal is selected from the group consisting of an equine, a feline, a canine, a bovine, a caprine, an ovine, and a murine.

Other embodiments and advantages of the invention are set forth in part in the description, which follows, and in part, may be obvious from this description, or may be learned from the practice of the invention.

### DESCRIPTION OF THE FIGURES

FIG. 1 Effect of Fibrinopeptide A in a natural and synthetic form on the survivability of mice to Ponto Toro infection. Activated Serum Fraction contains Goat Fibrinopeptides A and B as well as the Fragment of Compliment C3.

FIG. 2 Effect of PEGylated and non-PEGylated synthetic Fibrinopeptide A in an acute Experimental Allergic Encephalomyelitis mouse model.

FIG. 3 HPLC reading of the Bovine serum fraction embodiment of the invention. Peaks at 21.73 and 22.84 were both identified as SERIM A; Peaks at 22.59 and 23.28 were identified as SERIM B; the small peak at 20.13 seconds was identified as SERIM C.

FIG. 4 HPLC reading of the Equine serum fraction embodiment of the invention. Peaks at 21.32 and 18.30 were identified as SERIM A; peaks at 14.56 and 23.53 are SERIM B; the peak at 11.62 and 11.84 are SERIM C

FIG. 5 HPLC reading of the caprine serum fraction embodiment of the invention. The horse serum fraction contains the highest relative amount of Equine SERIM A (peak at 17.86). Other peptides were not identified in this specimen, but review of the protein databases shows no sequencing information for the other two SERIMs in the equine database.

FIG. 6 HPLC reading of the Human serum fraction embodiment of the invention. As can be seen, the human sample contains many more peptides than the animal samples. However, samples still correlate with the majority of the peptide mass. Peaks at 29.46 and 20.96 both correspond to Peptide A. Peaks at 25.27 and 30.41 correspond to peptide B, and the peak at 19.16 corresponds to peptide C.

### DESCRIPTION OF THE INVENTION

The human body has an amazing ability to heal following a severe traumatic injury. In considering the differences in response to injury between those suffering from severe trauma and those suffering minor injuries, three differences stand out: 1) Those involved in severe trauma have a markedly enhanced immune system response; 2) There is a relative minimization of swelling in the early stages of injury for those experiencing severe trauma; and 3) Severe Trauma creates a numbing effect, decreasing the pain felt by severe trauma patients when compared to those suffering more minor injury. The medical literature and the current medical paradigm attribute these findings to the “stress response” and the release of endogenous endorphins as part of this response. A group of peptides released during these types of injuries has been surprisingly discovered that are responsible for many of the benefits of this stress response. When these peptides are utilized in chronic diseases this response has tremendous benefits to the patient.

Herein are identified a number of cytokine activities of peptides, some of which have been previously identified as molecules but the cytokine activity has not been otherwise shown. In addition, certain molecules are characterized herein that were not previously identified as biologically active substances. While the sequences of certain peptides may be established, cleavages of these proteins and the releasing of biologically active peptides have not been previously described. These peptides fall in two classes: 1) those released as part of the clotting cascade, and 2) those released as part of the complement system. Many of the peptides released as part of the clotting cascade have been identified, but the cytokine mechanism of action has not been previously described or recognized. The peptides of the complement system have not been previously described as cleavages from the parent proteins, and their activity as cytokines also has not been previously described. The description herein discloses that these peptides are released in response to a break in the integument. Most any pathologic insult severe enough to cause damage to the walls of blood vessels will produce a similar release of these peptides.

During the initiation of the clotting cascade, many small peptides are released in the activation of the proteins which form the framework of a blood clot. These degradation products have always been considered relatively inactive peptides although some minor activities outside of the clotting cascade have been attributed to them. These peptides are present in the bloodstream just long enough to be further recycled, with half lives of only minutes. However, given the complex interaction between the various systems in other physiologic processes, degradation products from the clotting cascade have the ability to up-regulate the immune system, as the need for clotting typically coincides with exposure to pathogens. Also, cytokine activity usually occurs at very low doses. The ability of small volumes of these peptides to have a profound effect even with their very short half lives in the body is surprising. Although unexpected, this may be due to a need for an up regulation of the immune system when there is a breech in the walls of a blood vessel or of the integument.

Peptides of the invention have this ability to block the deposition of fibrin and associated material. This is a direct effect or part of the result of the immunomodulation and stimulation of the cytokine cascade, but the fact that Fibrinopeptide A either directly or indirectly results in the regulation of Fibrin deposition presents a major breakthrough in the management of both acute and chronic diseases. In addition, the complement system is activated in response to this same type of insult and the subsequent exposure to infectious agents. A previously unidentified peptide is released from the C3 protein of the complement cascade, and contributes to this immunomodulatory activity.

One embodiment of the invention is directed to an agent comprising a peptide containing a sequence identified in SEQ ID NOs. 1-5, 7-9, 11-13, 15, 16, or 20-22. Also included are peptides that comprise the sequence of Fibrinopeptide A or Compliment C3, and a sequence of a region of Fibrinopeptide A or Compliment C3 that is substantially homologous between species of mammals that produce Fibrinopeptide A or Compliment C3, respectively. The sequence may be derived from human or non-human sources. The invention is also directed to a sequence that contains one or more conservative amino acid substitutions of any of the aforesaid sequences. Preferably, the agent contains substantially no detectable Fibrinopeptide B. Preferably the agent further comprising a pharmaceutically acceptable carrier such as, for example, water, oil, edible oil, fatty acids, lipids, polysaccharides, cellulose, glycerin, glycol, and combinations thereof, and any of a number of conventionally used carriers such are disclosed in WO/010757 entitled “Pharmaceutical Composition” by J. Arch and N. Bowring (which is incorporated by reference). Preferable edible oil includes, for example, lemon oil, peppermint oil, or grape seed oil, or other natural oils and fatty acids derived from plants. Preferred agents are formulated for oral, transmucosal, parenteral, lymphatic, or intravenous administration such that the biologically active form of the agent is released into a system of a patient at a physiologically effective concentration. Also preferred is an agent which is purified from biological sources or synthetically manufactured, including both the peptide sequences themselves. The invention also includes nucleic acid sequences that encode these peptides.

Another embodiment of the invention is the agent described above and herein, that is a dietary supplement. The agents of the invention are safe for human and animal ingestion, and non-toxic at all effective dosages, and contain no endogenous endotoxin or other harmful materials or contaminants. Administration as a dietary supplement can be as the agent in a pure form, preferably transmucosally and more preferably suspended in a fatty acid, saccharide or polysaccharide, oil, or other carrier substance (e.g. as a liquid, gel, paste, powder, tablet, or pill) for immediate absorption by the mucosa of the mouth, such as under the tongue. As a dietary supplement, the agent can be administered to a patient or in association with other ingredients such as in a beverage or food product.

Another embodiment of the invention is directed to a pharmaceutical composition comprising Fibrinopeptide A or a fragment thereof, and a pharmaceutically acceptable carrier, wherein the Fibrinopeptide A or fragment thereof is at a therapeutically effective amount. Preferably the therapeutically effective amount is from 0.1 mg to 500 mg. Also preferred is the composition wherein the therapeutically effective concentration prevents deposition and stimulates resorption of fibrin within the extravascular spaces, such as is associated with coronary artery disease, and subintimal spaces in a patient. Preferably the composition is nontoxic at the therapeutically effective concentration and substantially free of detectable Fibrinopeptide B. The composition may caontain Fibrinopeptide A or fragment thereof that are derived from a human or non-human, but preferably mammalian sequence of Fibrinopeptide A. Mammals that express the non-human sequence of Fibrinopeptide A include an equine, a feline, a canine, a bovine, a caprine, an ovine, and a murine.

Another embodiment of the invention is directed to a method for treating or preventing a disorder of a patient comprising: providing a pharmaceutical composition comprising Fibrinopeptide A or Compliment C3, or a fragment of either, and a pharmaceutically acceptable carrier. Preferably the composition does not contain a detectable amount of Fibrinopeptide B, and the Fibrinopeptide A or Compliment C3, fragment of either, is derived from a mammal that is not a human; and administering a dose of the composition to the patient, wherein administration is transmucosal such that the Fibrinopeptide A or Compliment C3, fragment of either, achieves a therapeutically effective level within the lymphatic system of the patient within 5 minutes of administration. Preferably the patient is a human, and also preferably the disorder is vascular inflammation or coronary artery disease. The preferred single dosage of the composition contains from 0.1 mg to 10 mg of active ingredient, more preferably from 0.1 to 5 mg, and more preferably less than 1 mg. The administration may be on a periodic basis, and preferred administration comprises an initial administration of a single effective dose for a series of days, and a subsequently administered dose administered once every other day, more preferably once every few days, and more preferably once a week or even less frequently. Administration for all doses is preferably oral and transmucosal, such as under the tongue. Preferably the Fibrinopeptide A or fragment thereof stimulates the patient's cells to release cytokines IL1β, IL-10, and not IL-1, IL-4 or TNF\dot{α}. Another preferred aspect is for the activity of Fibrinopeptide B of the patient to be suppressed, such as, for example, by the administration of a Fibrinopeptide B binding agent. Binding agents include ligands, antibodies, or antibody fragments that are specific for Fibrinopeptide B, and, preferably, are non-toxic and include one or more substances (e.g. liquids or chemicals) that render the Fibrinopeptide relatively B inactive as compared with the activity of Fibrinopeptide A.

Another embodiment of the invention is directed to a method of preventing deposition of fibrin and also absorbing fibrin deposited within blood vessels and other areas of the body of a patient. These methods comprise: providing a pharmaceutical composition that comprises Fibrinopeptide A or Compliment C3, or a fragment of either, and a pharmaceutically acceptable carrier; and administering the composition to a patient such that the Fibrinopeptide A or Compliment C3, or fragment of either, is at a therapeutically effective level is achieved in the lymphatic system of the patient. Preferably the patient is a human and the Fibrinopeptide A or Compliment C3, or fragment of either, is derived from a mammalian sequence of the same molecule that is not a human. Administration of the composition is preferably directly to the lymphatic system by transmucosal administration, and comprises an initial administration and subsequently, a continued administration, and the continued administration is no more than once every few days such as once a week or even once a month.

Another embodiment of the invention is directed to a fraction of serum of a mammal wherein the fraction contains multiple components, is clarified of particulates, and substantially all components are within a defined molecular weight range. Methods to fractionate serum by molecular weight are well known and include dialysis with molecular weight cut-off membranes, centrifugation, and salt fractionation. The molecular weight range is preferably less than 3,000 Daltons, more preferably from about 5800 Daltons to about 2,500 Daltons, more preferably from about 1,000 Daltons to about 2,000 Daltons, more preferably from about 1,200 Daltons to about 1,800 Daltons, and more preferably from about 1,400 Daltons to about 1,800 Daltons. Preferably the mammal is an equine (horse), a canine (dog), a feline (cat), a bovine (e.g. cow, cattle, or bull), a caprine (goat), an ovine (sheep or lamb), or a murine (mouse), or may be any suitable mammal that produces Fibrinopeptite A or Compliment C3.

Another embodiment of the invention is directed to an agent comprising a peptide containing a sequence of SEQ ID NOs. 6, 10, 14, or 17-19, a sequence of Fibrinopeptide B, or a sequence of a region of Fibrinopeptide B that is substantially homologous between species of mammals that produce Fibrinopeptide B. Preferably the agent contains substantially no detectable amounts of Fibrinopeptide A. Preferably the agent further comprising a pharmaceutically acceptable carrier such as, for example, water, oil, edible oil, fatty acids, lipids, polysaccharides, cellulose, glycerin, glycol, and combinations thereof, or another conventional carrier such as is disclosed in WO/010757 entitled “Pharmaceutical Composition” by J. Arch and N. Bowring (which is incorporated by reference). A preferable edible oil includes, for example, lemon oil, peppermint oil, or grape seed oil, or anyother vegetable or fruit oil or fatty acid, or a plant oil, polysaccharide, or fatty acid. Preferred agents are formulated for oral, transmucosal, parenteral, lymphatic, or intravenous administration such that the biologically active form of the agent is released into a system of a patient at a physiologically effective concentration. Preferred administration is oral, under the tongue. Also preferred is an agent which is purified from biological sources or synthetically manufactured. The invention also includes nucleic acid sequences that encode these peptides.

Another embodiment of the invention is the agent described above and herein, that is a dietary supplement. The agents of the invention are safe for human and animal ingestion, and non-toxic at all effective dosages, and contain no endogenous endotoxin or other harmful materials or contaminants. Administration as a dietary supplement can be as the agent in a pure form, preferably transmucosally and more preferably suspended in a fatty acid, saccharide or polysaccharide, oil, or other carrier substance (e.g. as a liquid, gel, paste, powder, tablet, or pill) for immediate absorption by the mucosa of the mouth, such as under the tongue. As a dietary supplement, the agent can be administered to a patient or in association with other ingredients such as in a beverage or food product.

Another embodiment of the invention is directed to a method for treating or preventing a disorder of a patient comprising: providing a pharmaceutical composition comprising Fibrinopeptide B or a fragment thereof, wherein the composition contains substantially no detectable Fibrinopeptide A, and a pharmaceutically acceptable carrier, wherein the Fibrinopeptide B or fragment thereof, is derived from a mammal that is not a human; and administering a dose of the composition to the patient, wherein administration is transmucosal such that the Fibrinopeptide B or fragment thereof achieves a therapeutically effective level within the lymphatic system of the patient within 5 minutes of administration. Preferably the patient is a human, and the disorder is an auto-immune disorder, such as, for example, arthritis, Crohn's disease, Coeliac disease, diabetes mellitus type 1, Grave's disease, idiopathic thrombocytopenic purpura, psoriasis, scleroderma, systemic lupus erythematosus, or ulcerative colitis, or the disorder is a immunoregulatory disorder, such as, for example, an overactive immune system. The preferred single dose contains from 0.1 mg to 10 mg of active ingredient, or more preferably from 0.1 mg to 5 mg, or more preferably from 0.1 mg to 1 mg.

Another embodiment of the invention is directed to a fraction of serum of a mammal wherein the fraction contains multiple components, is clarified of particulates in a manner conventionally know, and substantially all components are within a molecular weight range of from about 800 Daltons to about 2,700 Daltons. Preferably the components are within the molecular weight range of from about 1,000 Daltons to about 2,500 Daltons, and more preferably from about 1,200 Daltons to about 1,800 Daltons. Preferably, the mammal is selected from the group consisting of an equine, a canine, a feline, a bovine, a caprine, an ovine, and a murine, but my be any suitable mammal that produces Fibrinopeptide B.

Fibrinopeptide A, natural or synthetic, regulates the Fibrin deposition in the extra-vascular space (both deposition of fibrin in this space and mobilization of fibrin deposits from this space) and thereby both control the progress of disease and ameliorate symptoms which result from this deposition. Accordingly, the invention is also directed to Fibrinopeptide A, natural or synthetic, to regulate the Fibrin deposition in the sub intimal space (both deposition of fibrin in this space and mobilization of fibrin deposits from this space) and thereby both control the progress of disease and ameliorate symptoms which result from this deposition.

A combination of Fibrinopeptide A and B has been utilized in therapeutic studies. But these studies have not differentiated the activity of one peptide from the other. In addition, most of the existing published research uses species-specific fibrinopeptides, thereby failing to demonstrate the cross species benefit. The activities of Fibrinopeptide A as an immunomodulator are shown herein. Also shown herein are the high interspecies homologous regions at the C terminus of the peptide.

Fibrinopeptides A and B act primarily on the immunologically non-specific phase of EAE development by reducing the severity of vascular permeability alterations through a pronounced direct anti-inflammatory response. This response ameliorates the acute inflammatory response in a disease process. This type of response is therefore not expected to greatly decrease the initial symptoms of an autoimmune attack, but over time to stop the attack and enhance the healing from the attack.

Fibrinopeptide A regulates both the deposition and resorption of fibrin, and extra-vascular fibrin. The processes described for obtaining the serum by any of the above methods produces a serum rich in the peptides of this invention. Considering no inhibitors of clotting are utilized in the majority of these preparations, coagulation naturally occurs immediately following removal of the blood from the donor animal or patient, releasing some or all of the peptides which are the object of this patent. Once released, these peptides undergo further natural processing to create the active peptide fragments. Since the filtration methods described in these patents should not eliminate these small peptides from the serum, and given the established efficacy of Fibrinopeptides A and B, these peptides are responsible for part or all of the therapeutic effect seen with all of these preparations.

Many chronic diseases exhibit the presence of fibrin deposits as an important pathologic part of disease progression. Preventing these deposits and eliminating the existing deposits represent important targets for therapeutics in these diseases. As evaluated herein, the benefits of these same peptides released as a response to traumatic injury was significant data demonstrating the ability of these peptides to prevent or slow the deposition of fibrin and stimulate the resorption of fibrin. These peptides are released in a staggered fashion in the activation of fibrin. Cleavage activates Fibrinopeptide A, which inhibits the deposition of fibrin, followed by Fibrinopeptide B, which promotes the deposition of fibrin. The combination however results in wound repair.

Utilizing mass spectrometry, a group of small peptides were isolated from serum after filtration of the sample to maintain only those substances that were preferably less than 3 kD in size. The vast majority of these peptides of the invention are by-products of the clotting cascade, although not previously utilized as therapeutic agents. The therapeutic activity of these peptides falls into three categories: 1) regulation of fibrin deposition and resorption of existing fibrin deposits; 2) modulation of the immune system from the passive mode seen in chronic disease to an active surveillance mode; and 3) anti-inflammatory activity.

The regulation of the deposition of fibrin into the extra-vascular space is recognized as an important potential target for therapeutics for a variety of diseases, including Lupus, Multiple Sclerosis, Atherosclerosis, Rheumatoid Arthritis, and Alzheimer's disease. In these (as well as many other diseases) the deposition of fibrin into the extra-vascular space is an important event in the progression of disease. While this fibrin deposition may not be the cause of a specific disease, the process started at the time of this deposition of fibrin is an essential pathologic element in the progression and tissue destruction caused by these diseases. This deposition also blocks the mechanisms the body usually utilizes to heal injured tissues. The ability of these peptides to block this deposition of fibrin has never been recognized as a potential therapeutic modality. In addition, the chronic deposition of fibrin is well established to prevent the normal healing of tissues, due to a cascade effect the presence of fibrin causes in these tissues. Peptides of the invention also trigger the resorption of these fibrin deposits, allowing the natural healing processes to resume in chronic diseases.

The immune cascade triggered by the injection of peptides of the invention demonstrates this type of combination Th1/Th2 response, as represented in the data demonstrating a consistent elevation of Interleukins 1B and 10, and inconsistent elevation of Interleukins 13, 5, 6, and 8 and TNF-α. These interleukins originate from immune cells (macrophages, monocytes and lymphocytes).

The anti-inflammatory response of peptides, Fibrinopeptide A and B, was first identified in a publication from 1978 (Ruhenstroth-bauer, et. al. U.S. Pat. No. 4,215,109). There is, however, a profound lack of any subsequent publication looking at this activity and specifically the lack of additional published studies in the Experimental Allergic (Autoimmune) Encephalomyelitis models. In addition, the authors did not identify the mechanism of an anti-inflammatory response, and did not identify the immune-stimulatory capacity of these peptides. As shown herein, this anti-inflammatory response is due in part to the ability of peptides of the invention to stimulate the release of the Th2 cytokines IL-10. While this response is meaningful in acute disease, the acute disease models are not designed to show the more significant benefit of this response in chronic diseases. With just Fibrinopeptide A, three study arms were run utilizing various doses of PEGylated Fibrinopeptide A. This modification was found to decrease any measurable activity of Fibrinopeptide A, demonstrating the importance of the degradation of Fibrinopeptide A in activation of this peptide.

In addition to this anti-inflammatory property of Fibrinopeptides A and B, the cascade initiated by these peptides increases the production of the pro-inflammatory cytokine IL-1B. IL-1B is an important mediator of the inflammatory response, and is involved in a variety of cellular activities, including cell proliferation, differentiation, and apoptosis (stimulated cell death). IL-1 stimulates thymocyte proliferation and differentiation, possibly by inducing IL-2 release, although elevation of IL-2 has not been demonstrated in our studies. IL-1 also stimulates B-cell maturation and proliferation, triggers the release of fibroblast growth factor and collagenase from synovial cells (a stimulator of other T and B lymphocytes). IL-1 has been identified as an endogenous pyrogen due to its ability to stimulate the release of prostaglandin. While the increase seen in IL-1B from these peptides does not seem to be sufficient to induce a pyrogenic response, the overall effect on the immune system is quite profound. The lack of pyrogenicity may also be in part due to the anti-inflammatory activity of IL-10 which is simultaneously stimulated.

As the integument is the first line of defense against infectious challenges, an immunomodulatory cascade occurs in response to any breech of that integument. The release of these peptides, in response to this type of breach, comprises at least a significant portion of this immunomodulatory activity. In the process of identifying the active ingredient of this immunomodulatory activity, the bioactive forms of these peptides are actually fragments of the previously describe peptides, and these fragments are much more active than the full peptide. To activate fibrin, Fibrinopeptides A and B are cleaved from the carboxyl termini of the fibrinogen subunits Aα and Bβ. Fibrinogen A and B then undergo further physiologic activation steps to become the potent immunomodulators. While many minor effects of these peptides have been observed, none are viewed as significant or as viable therapeutic options disclosed herein.

In addition to the release of the above clotting factors in response to a breach in the integument or as a response to an infectious insult, an immune cascade begins which heightens the ability of the immune system to seek out and destroy abnormal cells. This immune system stimulation can be broken down into two aspects, the innate and adaptive response. With introduction of abnormal cells into the body, the innate immune system responds rapidly to ward off the insult. One portion of this response is the activation of the complement cascade which activates a system to attack and destroy infectious organisms. In conjunction with this, a previously unidentified fragment of complement C3 protein is consistently present in the serum fractions from all mammals tested, suggestion the participation of this molecule in the immunomodulatory activity of these serum fractions. This protein causes a generalized stimulation of both the innate and adaptive portions of the immune system.

No previous indication of the cleavage site releasing this peptide has been previously identified, nor does the literature acknowledge that this fragment has any biological activity. This fragment is removed from the amine terminus of the portion of the C3 protein remaining after peptides C3 a-g have been removed. The remaining fragment is called Complement C3 alpha′ chain fragment 2. This fragment composes amino acids 1321-1663 of the Complement C3 protein, the remaining protein after C3f is cleaved and participates in the complement cascade. Following the cleavage of C3f (amino acids 1304-1320), it is demonstrated herein that additional enzymatic activity occurs. Cleavage of this peptide is species dependant, with an apparent homologous sequence at the amine terminus, but with differences at the carboxy terminus. The substitutions at the carboxy terminus have changed the cleavage site in humans compared to other mammals, but this change does not affect the activity of the molecule. In human serum, this peptide constitutes the sequence 1321-1336 of the C3 protein, with a sequence of SEETKENEGFTVTAEGK (SEQ ID NO. 16). In other species identified, this sequence contains a substitution of Arginine for Glycine at the 1329 position, with addition substitution at the 1333 position resulting in a change in the cleavage site. These substitutions therefore result in cleavage of a truncated peptide with the sequence SEETKENERFTV (SEQ ID NO. 7) in most other mammals. The homology between species varies slightly and therefore the sequence numbering varies between species. This alters the amino acid numbering of the location of this peptide. However, in each species this peptide is released as the next segment after the cleavage of complement C3f (C3g is located further toward the amine terminus than sequence C3f). With the minor exceptions described above, this latter sequence has strong homology in all of the species in which the full sequence for Complement C3 protein has been identified. In either case, this additional cleavage separates off a fragment containing significant immune system stimulation. This stimulation enhances the activity of both the innate and adaptive immune system allowing for a greatly enhanced activity.

While unrestrained, chronic stimulation of the immune system carries a significant risk of long term side effects as demonstrated with many of the other immune system stimulators, when this molecule is used in conjunction with the anti-inflammatory activity of activated fragment of Fibrinopeptide A the combination greatly enhances the immune system and the anti-inflammatory activity of Fibrinopeptide A essentially completely controls expected side effects. In addition to these findings, a synthetic peptide composed of the sequence constituting the amine terminus of imf-C3 and the carboxy terminus of af-FA has this dual action.

The strength of the data supporting the utility of this fragment is enhanced by the fact that a known substitution at amino acid 1320 of Arginine with Glutamine results in a C3 hypocomplementemia (C3 allotype C3′F02′) (Watanabe et al., 1993). This substitution which would be expected to alter the cleavage site causes a significant alteration in the immune system, resulting in a poor response to pathologic challenges, and demonstrating the essential presence of this molecule for normal immune response. This data also can be extrapolated to demonstrate the ability of this molecule to stimulate the innate immune system.

As each of the other protein fragments of Complement C3 is involved in the complement cascade and this peptide apparently is not. As part of the therapeutic serum fractions, this molecule has been shown herein to have a tremendous benefit in a variety of pathologic processes through the stimulation of the immune system. Utilizing the traditional nomenclature of the complement cascade, this protein would be Complement C3h. Since it does not participate in the complement cascade, this protein is referred to as immunomodulatory fragment of C3 (imf-C3), clarifying the type of activity of this peptide, which lies outside of the complement cascade.

The invention comprises obtaining a therapeutic component of serum or synthesizing the active peptides in the serum component through the process of de novo synthesis or fermentation, and utilizing these peptides to treat a host of infectious, inflammatory, neoplastic and autoimmune conditions. One embodiment of the invention is directed to a therapeutic modality which utilizes physiologically activated degradation products from the clotting cascade to activate a response in the immune system. In addition to the specific peptides described, similarly derived or synthesized peptides from the serum of other animals not specifically described in this patent are inclusive, as these peptides contain enough homology and similar characteristics to indicate that homologous peptides from other animals also will have the same therapeutic activity.

As embodied and broadly described herein, the present invention is directed to pharmaceutical compositions and methods for the preparation of a biologically active fraction of mammalian serum from animal blood to modulate the immune system, enhance the immune response, suppress the inflammatory reaction, and reduce the chronic deposition of extra-vascular fibrin of any other mammal under a variety of conditions. The biologically active fraction prepared according to the methods of the invention isolates peptides that provide for extensive new therapies. One embodiment of the invention is a method for providing the peptide, for example, by producing a biological active fraction of blood serum comprising the steps of: (i) withdrawal of blood from an animal; (ii) isolation of serum from said blood; and (iii) isolating a fraction containing these peptides. It is further preferred that the animal be a mammal, although the same characteristics are found in the serum of other animals such as a fowl. Preferred methods to produce a serum fraction containing these peptides include but are not limited to ultra filtration, HPLC separation, other forms of chromatography, tangential flow filtration, dialysis, centrifugation, electrophoresis, and others.

While raw serum allows for the therapeutic benefit, it is preferred that the serum is filtered to limit the other molecules given to the receiving animal. Ideally this results in elimination of any molecules larger than 6 kD, and preferably molecules larger than 3 kD. In a preferred embodiment of the method of the present invention the blood is arterial and/or venous blood. In a further preferred embodiment, the method further comprises the step of incubating said blood with thrombin, either physiologically or by the addition of thrombin in vitro. The method preferably comprises the step of lyophilization of said serum and the serum fraction is frozen and stored at −80° C. until near the time of usage. Alternatively, the lyophilized serum fraction can be suspended in an appropriate solution and administered orally as a dietary supplement to improve the normal function of the immune system.

### Treatment

1) Mother aspect of the present invention is the biologically active serum fraction which is producible according to the method of the present invention, or prepared in any way as to include the serum fraction of the present invention.
2) A further aspect of the present invention is the utilization of this serum fraction as a pharmaceutical preparation comprising this biologically active serum fraction according to the present invention combined with any of a variety of additional pharmaceutical grade additives to facilitate the utilization of the receiving animal. These additives may include fillers, carriers, binders, adsorbents, preservatives, diluents, etc. In a preferred embodiment of the pharmaceutical preparation of the present invention the preparation is formulated in a solution for subcutaneous or intramuscular injection. Other embodiments include the use of the invention as a topical preparation such as a gel, lotion or patch, a sublingual solution or preparation, a suppository, a lozenge, capsule or tablet, or the like.
3) Another aspect of the present invention is the use of the biologically active serum fraction of the present invention or of a pharmaceutical preparation of the present invention for the production of a medication for the treatment of acute bacterial infection in human and veterinary use.
4) Another aspect of the present invention is the use of the biologically active serum fraction of the present invention or of a pharmaceutical preparation of the present invention for the production of a medication for the treatment of chronic bacterial infection in human and veterinary use.
5) Another aspect of the present invention is the use of the biologically active serum fraction of the present invention or of a pharmaceutical preparation of the present invention for the production of a medication for prophylaxis against bacterial infection in human and veterinary use.
6) Another aspect of the present invention is the use of the biologically active serum fraction of the present invention or of a pharmaceutical preparation of the present invention for the production of a medication for the treatment of acute viral infection in human and veterinary use.
7) Another aspect of the present invention is the use of the biologically active serum fraction of the present invention or of a pharmaceutical preparation of the present invention for the production of a medication for the treatment of chronic viral infection such as HIV, HCV, HBV, HSV, HPV, etc., in human and veterinary use.
8) Another aspect of the present invention is the use of the biologically active serum fraction of the present invention or of a pharmaceutical preparation of the present invention for the production of a medication for the prophylaxis against viral infections such as those listed above in human and veterinary use.
9) Another aspect of the present invention is the use of the biologically active serum fraction of the present invention or of a pharmaceutical preparation of the present invention for the production of a medication for the treatment of parasitic diseases.
10) Another aspect of the present invention is the use of the biologically active serum fraction of the present invention or of a pharmaceutical preparation of the present invention for the production of a medication for the prophylaxis against parasitic diseases in human and veterinary use.
11) Another aspect of the present invention is the use of the biologically active serum fraction of the present invention or of a pharmaceutical preparation of the present invention for the production of a medication for the treatment of Autoimmune diseases including Rheumatoid arthritis, Systemic Lupus Erythematosus, Sceraderma, Mixed Connective Tissue Disease, Sjogren's disease, Psoriasis, Ankylosing Spondylitis and Reactive Arthritis, Behcet's Syndrome, Vasculitis, Sarcoidosis, Polyserositis, Amyloidosis, Chrohn's Disease, Ulcerative Colitis, etc., in human and veterinary use.
12) Another aspect of the present invention is the use of the biologically active serum fraction of the present invention or of a pharmaceutical preparation of the present invention for the production of a medication for the treatment of Neurologic disorders including Demyelinating Diseases (Multiple Sclerosis, etc.), Degenerative Diseases (Alzheimer's Disease, Parkinson's Disease, etc.), Neuropathies (Diabetic, idiopathic, Toxic, etc.) and other chronic nerve pain (RSD, etc.) in human and veterinary use.
13) Another aspect of the present invention is the use of the biologically active serum fraction of the present invention or of a pharmaceutical preparation of the present invention for the production of a medication for the treatment of Neoplastic Diseases including Carcinomas, Sarcomas, Leukemias, and Lymphomas in human and veterinary use.
14) Another aspect of the present invention is the use of the biologically active serum fraction of the present invention or of a pharmaceutical preparation of the present invention for the production of a medication for the treatment of inflammatory conditions of the musculoskeletal system in human and veterinary use.
15) Another aspect of the present invention is the use of the biologically active serum fraction of the present invention or of a pharmaceutical preparation of the present invention for the production of a medication for the treatment of Hyperimmune conditions such as mitigating the process of anaphylaxis and decreasing the intensity of seasonal allergies in human and veterinary use.
16) Another aspect of the present invention is the use of the biologically active serum fraction of the present invention or of a pharmaceutical preparation of the present invention for the production of a medication for the treatment of chronic wounds including chronic pressure ulcers, diabetic foot ulcers, etc in human and veterinary use.
17) Another aspect of the present invention is the use of the biologically active serum fraction of the present invention or of a pharmaceutical preparation of the present invention for the production of a medication for the treatment of other forms of chronic pain in human and veterinary use.
18) A further aspect of the present invention is the utilization of this serum fraction as a dietary supplement preparation comprising this biologically active serum fraction according to the present invention combined with any of a variety of additional food grade additives to facilitate the utilization of the receiving animal. These additives may include fillers, carriers, binders, adsorbents, preservatives, diluents, etc.

In a further preferred embodiment of the use of the present invention the medication is produced synthetically through the synthesis of these peptides. These synthetic peptides have the same biologic activity as the filtered fraction of mammalian serum. In this embodiment the invention is directed to the isolation and manufacture of the peptides comprising the sequence of SEQ ID NO 1-21 as well as conservative substitutions and modifications thereof. In a further embodiment of this invention these synthetic peptides are utilized for treatments 1-18 delineated above.

In a further preferred embodiment of the present invention, the medication is any peptide with the following characteristics of the activated fragments: 1) a N-terminus comprising 8 to 20 amino acids, 2) this portion typically contains a greater than average number of acidic amino acids, 3) a C-terminus containing the sequence FLAEGGGV SEQ ID NO 22), a homologous sequence, or a portion of this C-terminus comprising the sequence GGV (SEQ ID NO 21), and 4) an expected Arginine missing from the C-terminus when compared to cataloged peptides for fibrinopeptide A. This terminal sequence is highly conserved in mammalian species and is believed to be the active portion of the peptide.

Another embodiment of the present invention is a peptide sharing these characteristics or homologous structure to these three peptides possesses the same biologic activity as the invention, whether obtained from natural or synthetic sources. Preferably, these peptides represent conservative amino acid substitutions of one or more of the amino acids of Fibrinopeptide A or a fragment containing a conservative sequence thereof. Conservative substitutions are defined as those amino acid replacements that preserve the structure and functional properties of protein.

In this embodiment the peptide obtained from the fibrinogen alpha chain from any animal homologous to the amino acid sequence of the fibrinopeptide A in the homosapien sequencing data is included.

In a further embodiment of this invention peptides possessing these characteristics are utilized for treatments 1-18 delineated above.

Another embodiment of the invention is the process of removing blood from a patient, performing any purification/filtration process isolating a peptide with the above characteristics and then administering the peptide as an autologous injection to produce the biologic activity of the invention.

In this embodiment processing the serum may occur over a short time and the serum may be reinjected immediately, or the serum may be drawn in bulk and then small portions of the processed peptide containing product may be given at intervals over a prolonged timeframe.

In this embodiment the processing for autologous injection may occur by any of a variety of methods including Ultra filtration, HPLC separation, other forms of chromatography, tangential flow filtration, dialysis, centrifugation, electrophoresis, and many others.

In this embodiment the blood drawn is immediately placed in a centrifuge, the serum is separated and then processed or stored frozen until processing of the serum occurs. Dosage aliquots are stored frozen until immediately prior to injection.

In a further embodiment of this invention peptides thus processed for autologous injection are utilized for treatments 1-18 as delineated above.

Another embodiment of the invention is the production of antibodies or antibody fragments that are specifically reactive against peptides of the invention.

Another embodiment of the invention is directed to nucleic acid sequences, and sequences that hybridize thereto, that encode the peptides of the invention.

The following examples illustrate embodiments of the invention, but should not be viewed as limiting the scope of the invention.

### EXAMPLES

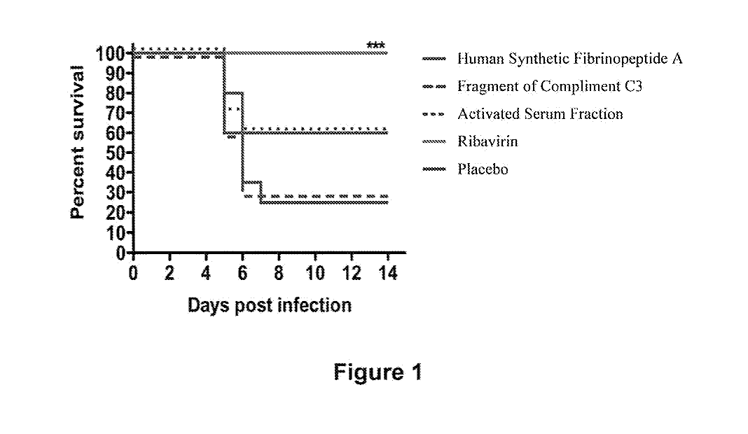

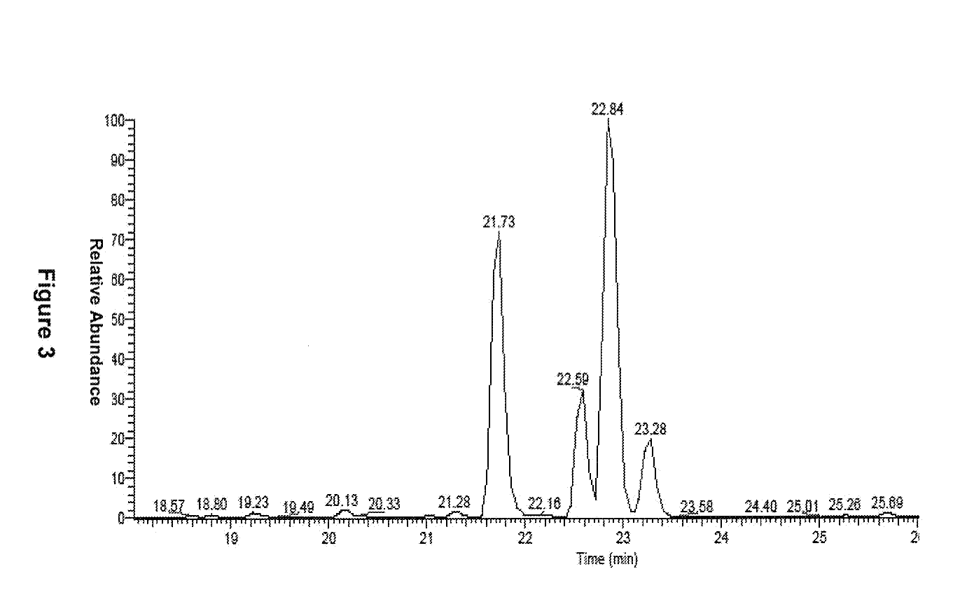

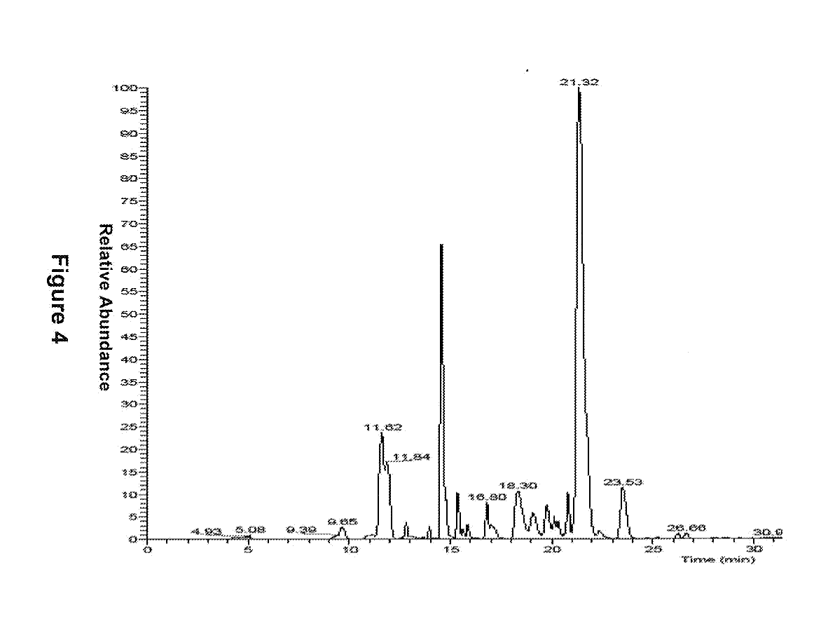

This invention is the product of a review of the available literature, analysis of mass spectrum data from ultra filtered fractions of human, bovine, feline, equine, and caprine serum, followed by establishing the fractions and the synthetic peptides as possessing the stated immunomodulation. The process of removing blood from an animal or human the clotting process is initiated unless a clot inhibitor is utilized. The peptides found in this ultrafiltered product are predominantly byproducts of the clotting cascade. It was surprising to find that the C-terminal Arginine in these previously defined peptides had been removed. This activity is due to the presence of Carboxypeptidase B. The presence of this enzyme in the bloodstream physiologically activates many peptides. The removal of the carboxy terminus arginine by this enzyme from fibrinopeptides A and B and imf-C3 is incidental, as this enzyme is present in the serum and performs this Arginine cleavage on a constant basis for molecules containing a carboxy-terminus Arginine. This removal activates these peptide into potent immunomodulators. The amount of the peptides that had the Arginine still attached was so minimal in the animal samples that it was hard to find the full peptide in the mass spectrometry data from any of the animals (See FIGS. 1, 2, 3 and 4). The only peptide that was isolated from all four mammal specimens was SEQ ID No 12, but the presence of this peptide in the bovine and equine specimen was minimal and may be associated with cross contamination. In the human sample (processed the same way) the amount of the peptide with the terminal Arginine still attached was far greater (See FIG. 4), indicating the process of removal of this Arginine by Carboxypeptidase B in human serum is a far less efficient process than occurs in animals.

In addition to the activated fragments of Fibrinogen described above, each of the samples except the equine sample contained the previously unidentified fragment of C3 complement described above. This fragment lies at the N-terminus of the complement C3c alpha' chain fragment 2, comprising 12-17 amino acids, depending on the species. Searching in three different data bases, this C3 complement does not appear to be sequenced in the horse, possibly accounting for the lack of identification of this peptide in that sample. In the other species analyzed, this fragment has considerable homology, especially of the Amine Terminus. The homologous segment in the human C3 compliment fragment also has not been identified as being cleaved from C3c alpha' chain fragment 2. This human C3 alpha chain fragment has a strongly homologous sequence at the N terminus, with the substitution of only one peptide through the first twelve amino acids. The data indicates that a higher quantity of this peptide in the caprine and bovine serum fraction analysis, possibly accounting for the preferential use of caprine serum in much of the available data. The activity of this molecule may account for the utilization of the goat as the primary source for animal serum in the serum fractions currently being used for therapeutic, as the presence of this peptide in the goat and cow appears to be significantly higher than was found in other species. The MASCOT search database used in conjunction with the Mass spectrometry results identified this peptide in the Bovine and Caprine samples as a sequence hit, but only identified it as a possible sequence hit in the other samples. The shorter goat and cow peptide was also found to have greater activity than the longer human naturally occurring peptide.

Although the fibrinopeptide B fragments seen in the samples from the various animals do not have any significant homology (a characteristic of both fibrinopeptide A and the described fragment of C3 compliment) fibrinopeptide B may still be an important part of some of the therapeutic benefits. This lack of any significant homology indicates the therapeutic benefit is most likely species specific, limiting the ability to use animal models to document benefit of human peptides. In fact, review of the sequencing information from various mammals shows that this area of the b chain of fibrinogen to have little homology even between closely related species (orangutan markedly different sequence from human fibrinopeptide B).

Once these peptides had been identified, a comparative analysis was performed to evaluate similarities in the serum fractions. Tremendous homology was found in the carboxy termini of peptide A and in peptide C, but no significant homology in peptide B between the species. Most of the interspecies activity is likely to reside in Peptides A and C.

As the data on anti-infective activity in serum fractions seemed the strongest, animal models for Ponto Toro and Influenza A H1N1 were used for these two viruses using three different specimens: 1) synthetic human activated fibrinopeptide A, 2) synthetic animal peptide imf-C3; and 3) a filtered lyophilized serum fraction from goat tested and found to contain all three peptides. While these substances did not perform as well as a direct anti-viral, the results did demonstrate improved survival of the treated animals when compared to the placebo group.

In this study several criteria were analyzed. These included liver, spleen, and serum virus titers; Serum alanine aminotransferase (ALT) determinations; livers and lungs were scored for hepatic icterus on day 3 of infection; daily weight measurement; Mean Day to Death; and overall survivability. The two groups treated with test articles containing Fibrinopeptide A performed identically. In these treatment groups 60% of the mice lived, while in the placebo group only 25% survived. This improvement was statistically significant for each of the Fibrinopeptide A treated groups independently (P value=0.03), and when these groups are combined to calculate the overall improvement with Fibrinopeptide A the statistical significance improved (P value=0.015). This increased survivability occurred even though there was no observable difference in any of the other disease criteria evaluated, suggesting no change in the ability of the virus to cause disease, but rather an increased ability of the organism to fight off a life threatening infection following one dose of peptide A. No measure of inflammation or fibrin deposition was performed in this study. While a difference exists between peptide A and the Ribavirin control, this difference was not statistically significant (P value=0.08).

These results demonstrate the therapeutic value of these peptides (particularly Fibrinopeptide A) in the infectious disease arena, with the ability to augment healing and decrease the duration of symptoms. As the body's normal response to infectious diseases results in a very pro-inflammatory state, even after the infection is removed this inflammatory state often causes persistent symptoms. Fibrinopeptide A has the ability to alleviate these symptoms and to therefore shorten the symptomatic phase of the disease without blocking the body's ability to fight off the infection. This effect may also be partially due to the ability of fibrinopeptide A to mobilize proteins (especially fibrin) out of the extra-vascular space.

In addition to this Ponto Toro study, a study was conducted to test these substances against Influenza A H1N1. In this study the control was low dose Ribavirin. All of the mice died in every group, indicating a more severe infection than anticipated.

Cytokine panels were evaluated from healthy volunteers after administration of these substances to more fully delineate the mechanism of action and therapeutic activity.

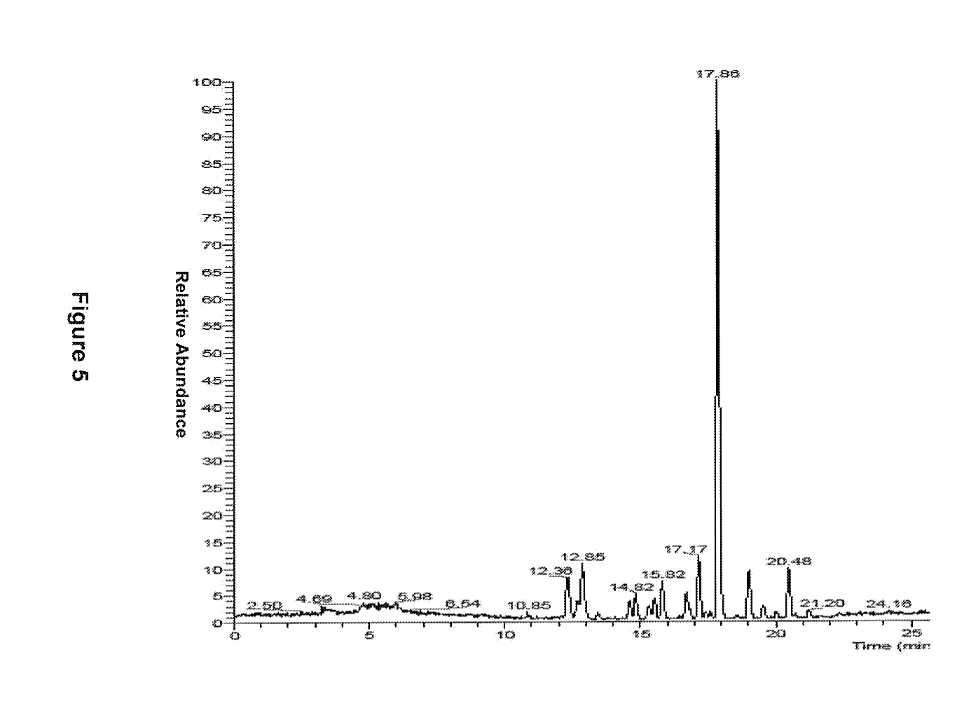

In an effort to document the effect of these peptides, a filtered serum sample containing peptides of the formulation of this invention (see HPLC tracing of the goat filtration fraction, FIG. 5) was administered to a healthy volunteer, initially utilizing the preparation from a goat. A cytokine 12 panel was obtained immediately prior to this administration, and then at intervals following administration (15 minutes, 1 hour, and 3 hours following administration; see Table 1). Based on published studies, a shift in the cytokine panel during these intervals was expected, but far less effect was observed. Another much higher dose of an autologous preparation was administered to the same healthy volunteer 5 weeks later. Note in the last column of Table 1 the marked difference in the initial levels of Interleukins 10, 13, and 1β. In addition, there is a slight increase in the level of Interleukin 2 receptor. Given the subtle changes in the first set of data, the subtle shift in the cytokine panel initially seen continued to escalate over an extended period of time.

TABLE 1
Cytokine 12 Panel following administration of Caprine peptides of this
invention

| TEST | | Pre | 15 min | 1 hour | 3 hours | 5 weeks |
|---|---|---|---|---|---|---|
| IL-2 | nl: 0-12 | 0 | 0 | 0 | 0 | 0 |
| IL-2R | nl: 0-1033 | 364 | 420 | 377 | 416 | 593 |
| IL-12 | nl: 0-6 | 0 | 0 | 0 | 0 | 0 |
| IFN-γ | nl: 0-5 | 0 | 0 | 0 | 0 | 0 |
| IL-4 | nl: 0-5 | 1 | 1 | 1 | 1 | 0 |
| IL-5 | nl: 0-5 | 0 | 0 | 0 | 0 | 0 |
| IL-10 | nl: 0-18 | 4 | 7 | 6 | 8 | 19 |
| IL-13 | nl: 0-5 | 4 | 7 | 5 | 5 | 30 |
| IL-1B | nl: 0-36 | 7 | 12 | 7 | 9 | 32 |
| IL-6 | nl: 0-5 | 0 | 0 | 0 | 0 | 0 |
| IL-8 | nl: 0-5 | 0 | 0 | 0 | 0 | 0 |
| TNF-α | nl: 0-22 | 0 | 0 | 0 | 0 | 0 |

*Above timed tests were obtained immediately before injection with a caprine serum fraction containing the peptides of FIG. 5, and then at 15 minutes, 1 hour, and 3 hours after injection. The last set of data (5 weeks) was from a separate test on the same human subject and was prior to any intervention.

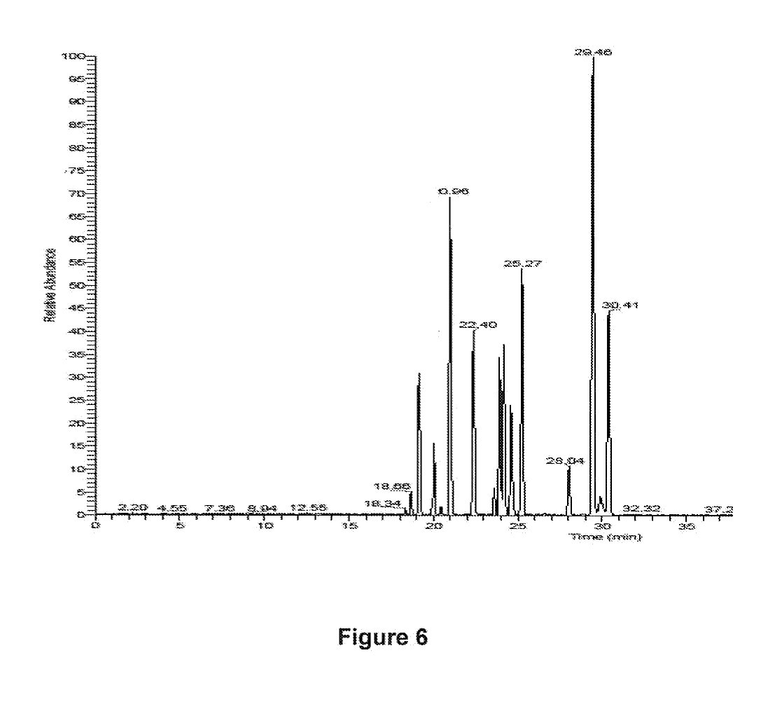

TABLE 2
Timed Cytokine 12 Panels following administration
of an autologous preparation of peptides of FIG. 6.

| | | | 15 | 1 | 3 | 6 | 12 | 24 |
|---|---|---|---|---|---|---|---|---|
| TEST | | Pre | min | hour | hours | hours | hours | hours |
| TNF-α | (0-22) | <5 | <5 | <5 | <5 | <5 | 6 | <5 |
| INF-γ | (0-5) | <5 | <5 | <5 | <5 | <5 | <5 | <5 |
| IL-5 | (0-5) | <5 | <5 | <5 | <5 | <5 | <5 | <5 |
| IL-13 | (0-5) | 30 H | 28 H | 28 H | 30 H | 30 H | 24 H | 23 H |
| IL-12 | (0-6) | <5 | <5 | <5 | <5 | <5 | <5 | <5 |
| IL-4 | (0-5) | <5 | <5 | <5 | <5 | <5 | <5 | <5 |
| IL-10 | (0-18) | 19 H | 15 | 18 | 13 | 19 H | 28 H | 13 |
| IL-1β | (0-36) | 32 | 28 | 24 | 24 | 23 | 50 H | 28 |
| IL-6 | (0-5) | <5 | <5 | <5 | <5 | <5 | <5 | <5 |
| IL-8 | (0-5) | <5 | <5 | <5 | <5 | <5 | <5 | <5 |
| IL-2 | (0-12) | <5 | <5 | <5 | <5 | <5 | <5 | <5 |
| IL-2R | (0-1033) | 593 | 587 | 539 | 513 | 547 | 591 | 537 |

The second test was designed to look at the cytokine panel extending out only for 24 hours. In this second set of data, (Table 2) note the shift in these same cytokines occurring in the 12 hour test. Again, in the 24 hour period evaluated, the shift is quite subtle. In general, these shifts would not be expected to produce rapid effects in the disease states of individuals, and they therefore do not fully explain the mechanism of action of this group of peptides. They do demonstrate the bioactivity of these peptides and as the response is consistent with clinic benefit, they validate use of the peptides.

Note not only the same shift described above with the first set of Cytokine 12 profiles, but also the prominent difference between the initial values over five weeks after the initial evaluation was done. This demonstrates a prominent shift in the cytokine panel that persists for several weeks after the initial dose was given. This shift is most notable in the levels of IL-13 and IL-10, Th2 cytokines with a very prominent anti-inflammatory activity. In addition, note that the levels of IL-1 B are considerably higher both at the pre dose value and a spike which occurs 12 hours after injection. IL-1B is considered a proinflammatory cytokine due to the stimulation of immune system cells following injection. In addition, at 12 hours there is a mild rise in the level of TNF-α, also potentially demonstrating an enhancement of the immune system as a significant part of the mechanism of action.

After getting back the results of the Ponto Toro test and seeing no results from imf-C3, a healthy volunteer was given synthetic imf-C3 and cytokine panels were followed for 24 hours. These cytokine panels did not demonstrate in any changes over the 24 hour time frame. Giving just Fibrinopeptide A does not result in an elevation of Interleukin-13, and just giving Fibrinopeptide A alone may result in long term stimulation of a pro-inflammatory response, which does not occur when giving the blend of these peptides.

Peptide was suspended in an appropriate medium and a healthy volunteer took the preparation daily for two weeks, drawing blood for a cytokine 12 panel every five days. Due to the size of the molecules absorption from the mucosal route seemed unlikely, and the rapid digestion of peptides in the stomach would eliminate their efficacy in a true oral route. This same healthy volunteer had lab work drawn right before receiving an IV dose of synthetic Fibrinopeptide A. The IV dose was expected to have a more profound therapeutic effect, since treatment was simulating a normal intravascular biologic process. It was surprisingly discovered that the initial sample drawn prior to IV injection of synthetic Fibrinopeptide-A demonstrated a more significant increase in the Interleukin-10 and Interleukin-1b levels than seen in any of the previous tests. (Table 3).

TABLE 3
Cytokine 12 Panels timed after an IV dose of
50 mg of Synthetic Fibrinopeptide A

| TEST | | Pre | 10 Hours | 14 hours | 60 hours | 30 days |
|---|---|---|---|---|---|---|
| IL-2 | nl: 0-12 | <5 | <5 | <5 | <5 | <5 |
| IL-2R | nl: 0-1033 | 550 | 475 | 492 | 425 | 546 |
| IL-12 | nl: 0-6 | <5 | <5 | <5 | <5 | <5 |
| IFN-γ | nl: 0-5 | <5 | <5 | <5 | <5 | <5 |
| IL-4 | nl: 0-5 | <5 | <5 | <5 | <5 | <5 |
| IL-5 | nl: 0-5 | <5 | <5 | <5 | <5 | <5 |
| IL-10 | nl: 0-18 | 96 | 87 | 87 | 74 | 22 |
| IL-13 | nl: 0-5 | N/A | N/A | N/A | N/A | <5 |
| IL-1β | nl: 0-36 | 52 | 52 | 54 | 54 | <5 |
| IL-6 | nl: 0-5 | 13 | 11 | 10 | 7 | <5 |
| IL-8 | nl: 0-5 | <5 | <5 | <5 | <5 | <5 |
| TNF-α | nl: 0-22 | <5 | <5 | <5 | <5 | <5 |

Note the elevation in the initial lab test of the IL-10, IL-1b, and IL-6 tests, occurring prior to the IV dose and 4 weeks after oral dosing.

In addition to these elevations, a mild elevation of Interleukin-6 was also observed. Interleukin-6 is a pro-inflammatory cytokine and is a very strong stimulator of the innate immune system. Equally surprising, the IV administration of a dose 10 times stronger than the subcutaneous dosage given previously led to no appreciable response. The most significant of these tests is the blood drawn one month after the IV dose was given. In this test the marked elevation of at least IL-10 and IL-1β, which had been present in each of the other dosages given by any route, was observed. This data, especially when taken together with the Experimental Autoimmune Encephalomyelitis date, discussed below, strongly indicates the importance of this molecule in the lymphatic system as a primary site of activity. In the blood stream these molecules are rapidly degradated and perhaps they do not even have the ability to stimulate the cells or molecules necessary to begin the process of resorption of fibrin and stimulation of the immune system. This location for primary activity also explains the stimulation of the adaptive immune system over the innate immune response, as the lymphatic system is more involved in the adaptive response, while the vascular system is more involved in the innate response. The response to orally administered Fibrinopeptide A also solidifies this location of activity as transmucosal absorption occurs almost exclusively through the lymphatic route, occurs rapidly for those molecules which are absorbed, and the oral submucosal region (especially the sublingual region) has an extensive lymphatic drainage with rapid access to the cells which would produce this type of response while at least partially being shielded from the enzymes that inactivate the peptides of this invention. Since chronic fibrin deposition in the extravascular space is always pathologic and pro-inflammatory, it is not surprising that patients have a mechanism triggered by this deposition to aid in the removal of these substances. This patent demonstrates that this mechanism occurs through the release of Fibrinopeptide A in conjunction with the initiation of this deposition, triggering the delayed resorption of these fibrin deposits. These results also demonstrate that a significant portion of the activity of fibrinopeptide A occurs at least 3 days after treatment with fibrinopeptide A, an acceptable timeframe to assure the clot has matured and is infiltrated with fibroblasts before the fibrin removal begins. The persistent activity of a single dose of Fibrinopeptide A, elevating the levels of these cytokines for over a month, demonstrates the importance of complete removal of this pro-inflammatory protein from the extravascular space. By signaling the need for removal from the extravascular space, the same process should occur for similar chronic deposits in the subintimal space.

TABLE 5
Oral Daily dosing with 3 mg Fibrinopeptide A
(Limited prior exposure to this peptide)

| TEST | | Pre | 1 Week | Off 1 Week |
|---|---|---|---|---|
| IL-2 | nl: 0-12 | <5 | <5 | 6 |
| IL-2R | nl: 0-1033 | 513 | 423 | 147 |
| IL-12 | nl: 0-6 | <5 | <5 | 6 |
| IFN-γ | nl: 0-5 | <5 | <5 | 5 |
| IL-4 | nl: 0-5 | <5 | <5 | <5 |
| IL-5 | nl: 0-5 | <5 | <5 | 5 |
| IL-10 | nl: 0-18 | 24 | 137 | 28 |
| IL-13 | nl: 0-5 | <5 | <5 | 8 |
| IL-1β | nl: 0-36 | <5 | 30 | <5 |
| IL-6 | nl: 0-5 | <5 | <5 | 5 |
| IL-8 | nl: 0-5 | <5 | <5 | 8 |
| TNF-α | nl: 0-22 | <5 | <5 | <5 |

The efficacy of oral (sublingual) Fibrinopeptide A was further demonstrated in two patients as displayed in Tables 5 and 6. One of these patients was the same patient previously utilized, while the other was relatively neve to any previous Fibrinopeptide A treatment. The patient had taken two doses orally approximately seven weeks prior to this treatment, no lab work had been obtained with this prior dosing. This patient took one preparation of Fibrinopeptide A orally daily for one week, and then did not take any further Fibrinopeptide A and blood was checked again after the patient had not taken any Fibrinopeptide A for one week.

Note the response in the IL-10 and IL-1b in this patient similar to the other patients. However, this patient return back toward baseline values much more rapidly than the other patients, accentuating the variability expected in this type of response from one patient to another. This also validates the efficacy of oral administration to achieve an anti-inflammatory/immunomodulatory benefit.

The other patient had received Fibrinopeptide A one month prior beginning oral dosing with Fibrinopeptide A. This initial cytokine panel shows the same changes seen in previous studies with Fibrinopeptide A and again results in a significant change in the Cytokine panel for well over a month. However, as shown in Table 6, the Cytokine panel had return to almost normal at the conclusion of the study. This again elucidates the lack of effect of Fibrinopeptide A when given IV and the importance of utilizing a lymphatic route when treating with Fibrinopeptide A, and probably the others as well.

TABLE 6
Cytokine 12 panels following oral administration

| | | | | | | 2 weeks |
|---|---|---|---|---|---|---|
| | | Pre and 1 | | | | back on |
| | | month | | | 2 Weeks | FPA |
| | | after | 1 | 2 | off | (different |
| TEST | | IV Use | Week | weeks | FPA | carrier) |
| IL-2 | nl: 0-12 | <5 | <5 | 7 | 7 | 7 |
| IL-2R | nl: 0-1033 | 546 | 504 | 254 | 255 | 268 |
| IL-12 | nl: 0-6 | <5 | <5 | 6 | 6 | 6 |
| IFN-γ | nl: 0-5 | <5 | <5 | 5 | <5 | 5 |
| IL-4 | nl: 0-5 | <5 | <5 | <5 | <5 | <5 |
| IL-5 | nl: 0-5 | <5 | <5 | 5 | 6 | 6 |
| IL-10 | nl: 0-18 | 22 | 141 | 183 | 230 | 204 |
| IL-13 | nl: 0-5 | <5 | <5 | 6 | 8 | 9 |
| IL-1β | nl: 0-36 | <5 | 30 | 83 | 37 | 48 |
| IL-6 | nl: 0-5 | <5 | <5 | 77 | 66 | 63 |
| IL-8 | nl: 0-5 | <5 | <5 | 8 | 8 | 8 |
| TNF-α | nl: 0-22 | <5 | <5 | <5 | <5 | <5 |

The data in table 6 further illustrates the up regulation of all aspects of the immune system in response to this persistent exposure to synthetic activated fibrinopeptide A through a lymphatic administration. Note the stimulation of the adaptive immune system through the marked elevation of IL-1β (a finding consistently demonstrated in the other cytokine panels), and also now a significant elevation of IL-6, a very strong stimulator of the innate immune system.

Another study was conducted to determine how this molecule could be structurally enhanced to maximize the benefit. In this study, several peptides were identified whose activity was markedly improved by protecting them from degradation. This was typically accomplished through the addition of a long molecule to the inactive portion of the peptide. The carboxy terminus of activated Fibrinopeptide A was considered to be the active portion of the peptide, the carboxy terminus was PEGylate (addition of Poly Ethylene Glycol). This molecule was used to perform an EAE study. This study was designed to mimic the study done by Ruhenstroth-Bauer in 1981 utilizing two of these peptides, and showing some albeit mild benefit. The synthetic PEGylated peptide was expected to perform considerably better than the non-PEGylated peptide. Synthetic non-PEGylated peptide A was used as a positive control to compare the PEGylated verses non-PEGylated peptides. The medications were administered subcutaneously, due to this route having some efficacy in the cytokine profiles.

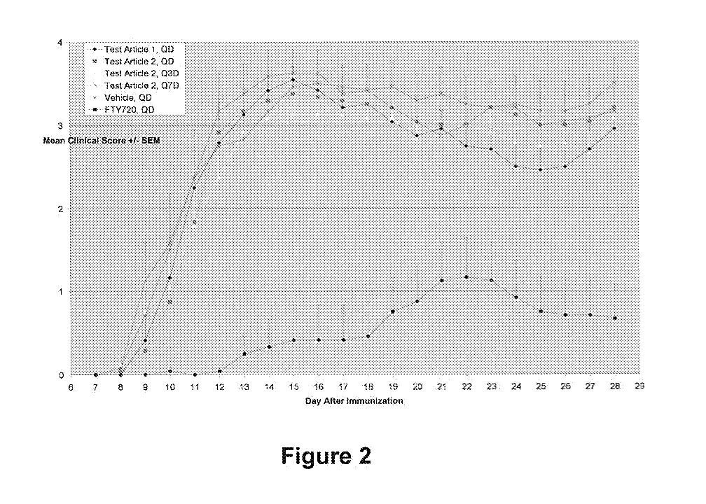

The PEGylated peptide had little or no response. As demonstrated from the graph (FIG. 2), none of the test article treated groups showed significant differences in EAE development from the vehicle-treated mice. Test article 2 (PEGylated peptide) dosed daily, every 3 days and weekly never even approached statistically significant benefit.

During the first 22 days of the study EAE development in mice treated with test article 1 (daily non-PEGylated peptide A) was very similar to the vehicle-treated mice. Then, mice in this group started recovering, while the vehicle-treated mice showed worsening of disease. This difference in disease severity between these groups did not reach statistical significance (p<0.1). Disease worsened in test article 1-treated mice on days 27 and 28 and became very similar to disease severity to the mice in the vehicle-treated group. It is not clear if this difference in disease severity was serendipitous or a result of some efficacy of non-PEGylated peptide A, but as it did not reach statistical significance.

First, since the PEGylated peptide had no activity, this indicated an additional cleavage was necessary to fully activate the peptide. PEGylation may have prevented migration to the site of greatest benefit—the lymphatic or at least extravascular compartment. PEGylating the peptide increased its size from approximately 1500 kD to greater than 30,00010, a size difference that would definitely be expected to prevent it from easily crossing into the extravascular space.

Secondly, this study demonstrated the effect of this peptide does not block the initial attack of an autoimmune disorder. The first dose was given 24 hours prior to induction of EAE. Symptoms were expected to be worse with administration of the non-PEGylated peptide, as the production of auto-antibodies would initial be increased. The long term benefits are still expected to improve all disorders of this type as the IL-1b will increase the surveillance and elimination of B-Cells producing auto-antibodies. The subtle non-statistically significant improvement seen from days 22-26 could be due to the decrease in inflammation and fibrin deposition in the extravascular space.

This study also demonstrated the possibility of enhancing peptide activity by shortening the size to allow migration into the extravascular space, indicating that the cleavage/degradation products of the peptides will also produce activity.

Currently the medical and scientific communities have adopted a belief that if a little of a given substance is good, a lot of that substance is usually better. The type of changes seen in the cytokine panel may be inadequate to qualify the utilization of these peptides as a therapeutic. However, in nature the harsh adjustments in homeostasis associated with most medications do not exist. These peptides through this type of gentle correction have the ability to restore the normal functional state of the immune system by shifting the immunologic state back from a permissive to an active response. This change has the ability to enhance the body's surveillance against infectious diseases, and malignant tumor cells, eliminate cells producing auto-antibodies, stop the harmful aspects of the mechanisms of inflammation, stimulate the absorption and elimination of harmful molecules deposited outside of the vascular lumen, and decreases the chronic stimulation of sensory neurons which result in chronic pain.

The mechanism of action involves, at least: Enhanced Immunity; Decreased Inflammatory Response; Prevention of deposition and stimulation of resorption of Fibrin in the extravascular and subintimal spaces

### Enhanced Adaptive Immunity

The mammalian immune system is divided into two types of immune response. The innate immune system is described as protective against acute insult, protecting the organism until the adaptive immune system can take over. The activity of the peptides activated fragment of Fibrinopeptide A (af-FA), activated fragment of Fibrinopeptide B (af-FB), and immunomodulatory fragment of C3 Complement (imf-C3) indicates utilization of a different division of the immune response more along the lines of an active verses a permissive immune response. Under this concept, the active immune response enhances the organism's capacity to recognize, seek out and destroy any pathogen or cell containing foreign or abnormal characteristics and destroy these cells through the stimulation of various cytotoxic and phagocytic cells, and through the stimulation of B and T cells. This partially accomplished through a cytokine cascade, initiated by the release of cytokines that are generally accepted as proinflammatory. However, when stimulated through administration of these peptides, the response seen clinically is actually an anti-inflammatory/immune stimulatory response. This response is due to the ability of these peptides to greatly enhance the localized and destructive ability of these cells while mitigating the damage done to the cells surrounding the foreign object or pathologic cells. This mechanism allows for a much more localized and direct activity against any pathogen by greatly enhancing the immune system's ability to destroy pathogenic insults while minimizing the systemic and even localized destructive reaction. Through the localized activity of NK lymphocytes, macrophages and T lymphocytes, the organism is able to mount a very aggressive attack on pathogens through a multifaceted reaction without the tissue destruction which usually accompanies these types of reactions.

The timed cytokine panels (Tables 1 and 6) provide insight into an explanation for this activity. The immediate and persistent elevation of cytokine IL-1β demonstrates the initiation of a cytokine cascade immediately after injection. This indicates a receptor on the surface of monocytes and/or macrophages, as these are the primary source of IL-1□ production. IL-1□ increases the presence of adhesion factors, enabling transmigration of Neutrophils and other leukocytes to the site of infection without stimulating the release of cytotoxic substances from these cells. This process carries a tremendous benefit when localized at the source of a breach of the integument. On a systemic basis the increased surveillance this process stimulates augments the body's ability to seek out and destroy any pathogens, abnormal cells, or even deposit of abnormal proteins. While IL-1β has previously been implicated in harmful neuro-inflammation, recent evidence contradicts this theory and demonstrates a definite benefit of the stimulation of IL1β in brain disease. (Shaftel, 2008)

### Decreased Inflammatory Response

In addition to the immediate rise in IL-1β, there is a rapid elevation in the expression of Interleukin-1 0 (IL-10). IL-10 is recognized as a pleotropic cytokine, with predominant anti-inflammatory effects. IL-10 is produced primarily by monocytes, again indicating a mode of action with primary effect on a monocyte receptor. IL-10 down-regulates the expression of Th1 cytokines, explaining the profound and rapid anti-inflammatory activity of these peptides. This anti-inflammatory activity in the presence of activation of T killer lymphocytes, NK cells and Neutrophils explains the decreased symptoms experienced by an organism while enhancing the ability of the organism to eliminate any pathogenic challenges. IL-10 also enhances B cell survival, proliferation, and antibody production. In the presence of IL-1β, the potential for this to result in auto-immune disease is eliminated by the increase in T-Killer lymphocytes stimulated by IL-1β, eliminating cells producing auto-antibodies.

In addition, IL-10 counteracts the inflammatory effect of mast cells, mitigating the effect these cells have in hypersensitivity reactions. IL-10 also plays a significant role in the differentiation and function of the T regulatory cell, which plays an important role in the direction of the immune responses and tolerance. IL-10 has been shown to stimulate angiogenesis, an important part of wound healing (Dace et al 2008).

Working in conjunction with IL-10, IL-13 levels inconsistently rise following an injection of these peptides. IL-13 is produced by activated T lymphocytes that inhibit inflammatory cytokine production induced by bacterial endotoxin. It also stimulates gamma-interferon production by natural killer cells, enhancing the effect of interleukin-2. IL-13 is best known for induction of reactive airway disease, but despite the roll of IL-13 in the activation of the immune system by these peptides, no hypersensitivity reactions were seen from this activation. To the contrary, all of the available literature supports the use of these peptides in the treatment of the hypersensitivity reactions. This is likely due to the combination activation of IL-10 in conjunction with this IL-13 up regulation, and is strong evidence of the role of IL-13 in the bodies attempt to stop an asthma attack rather than the presence of IL-13 being causative. Whether this response is important in the activity of these molecules will require additional research. It may also be that the presence of the combination causes this rise while limiting the rise of some of the other proinflammatory cytokines seen with the oral administration of Fibrinopeptide A in Table 6.

### Prevention of Deposition and Stimulation of Resorption of Fibrin in the Extravascular and Subintimal Spaces

Fibrinopeptide A has the ability to stimulate the absorption of Fibrin from the extra-vascular space. Several studies imply the ability of fibrinopeptide A to mobilize the already deposited fibrin. In two of these studies, Ancrod was utilized to increase the uptake and metabolism of fibrinogen from the blood stream. In both of these studies, it was concluded that producing a hypofibrinogenemic state increased the body's resorption of fibrin from the extra-vascular state. This method of inducing hypofibrinogenemia has the side effect of releasing Fibrinopeptide A. Ancrod, like Thrombin, cleaves the Arg-Gly bond, releasing Fibrinopeptide A from the Aα chain of Fibrinogen. Fibrinopeptide A is then further activated by removal of the terminal Arginine. Unlike Thrombin, Ancrod does not cleave the Arg-Gly bond connecting Fibrinopeptide B to the Bβ chain of Fibrinogen. This highly specific activity releases Fibrinopeptide A and results in rapid uptake of the remaining Fibrinogen fragment (desAA-fibrin monomer) by the liver, resulting in hypofibrinogenemia.

Ancrod is now a generally accepted way of experimentally producing hypofibrinogenemia in animal models (trade name VIPRINEX®). The most recent of these indications was for the treatment of acute ischemic stroke. The data indicates that a great deal of the therapeutic benefit of this treatment is produced by simply giving Fibrinopeptide A, markedly reducing the swelling, decreasing vascular leakage, and encouraging fibrinolysis without increasing the risk of Intracranial Hemorrhage or other coagulation disorder.

In addition, this hypofibrinogenemic state is wrongly theorized to be responsible for a wide range of therapeutic effects. This includes significant improvement in the symptoms of Lupus Erythematosus (Cole et. al. 1990) in which Ancrod treatment markedly slowed the progression of renal disease and procoagulant activity, resulting in marked improvement in survival with Ancrod therapy. In another study, Ancrod therapy was utilized to treat Glomerulonephritis (Kim, et. al. 1988). They evaluated the functional, immunogenic and histopathologic effects of Ancrod fibrinolysis in acute glomerulonephritis. Their findings for short term improvement (14 day study) demonstrated improvement in all three areas investigated. They also demonstrated an increase toward normal in C3 and C4, a decrease in serum Igs, a decrease in Gamma Globulin and anti-dsDNA antibody, and a decrease in glomerular C3 and Ig deposits, suggesting an improvement in immunologic factors in patients with Lupus nephritis. The histopathologic results from this study demonstrated the prevention of further glomerular sclerosis in these patients.

Similar findings where recently demonstrated in Alzheimer's disease by Paul et. al. in 2009 at Rockefeller University. In their study they used a transgenic mouse model of Alzheimer's disease and identified fibrin deposition to be an important participant in the development of b-amyloid neurofibrillary tangle pathology and blood-brain barrier permeability. Utilizing three experimental models they demonstrated this causative activity: 1) Mice with genetically decreased functional plasminogen have increased neurovascular damage, while mice with genetically decreased functional fibrinogen have decreased blood-brain barrier damage; 2) Treatment of Alzheimer's Disease mice with a plasmin inhibitor exacerbates pathology, while removal of fibrinogen with ancrod treatment slows progression of inflammation surrounding β-amyloid lesions; and 3) Pretreatment with ancrod slowed pathologic progression from plasmin inhibition. These studies implicate fibrin in the neuroinflammatory process of Alzheimer's disease. While the primary cause of Alzheimer's disease still appears to be β-amyloid protein, the disease does not seem to progress without the deposition of fibrin this protein induces. By slowing or blocking the process of fibrin deposition, or stimulating the resorption of deposited fibrin, Alzheimer's disease progression can be stopped and the symptoms ameliorated.

In all of these studies, Ancrod was utilized to produce hypofibrinogenemia. The researchers postulate hypofibrinogenemia is responsible for these positive effects, as well as their side effects. The data demonstrates the positive results can be obtained by only utilizing Fibrinopeptide A, without the side effects expected from this type of marked compromise of the coagulation cascade. In each of the referenced studies, the investigators failed to recognize the ability of the released fibrinopeptide A to stimulate the reuptake of deposited fibrin, prevent further deposition of fibrin, and markedly attenuate the inflammatory response in both the acute and chronic phases of these diseases.

This data demonstrates the ability of Fibrinopeptide A to improve these diseases by slowing the deposition of Fibrin in and stimulating the resorption of Fibrin from the extra-vascular and subintimal spaces. One mechanism is the activation of Tissue Plasminogen Activator, Urokinase Plasminogen Activator, or the inhibition of Tissue Plasminogen Activator Inhibitor. In addition Protein C plays an important role. As the overall activity favors the resorption of fibrin deposition, activation of Urokinase plasminogen activator is the mechanism to explain the benefit seen in the removal of fibrin deposition of chronic disease. In addition, Fibrinopeptide A has the ability to slow the migration of all serum proteins from the vascular space into the extra-vascular space. This effect is most likely due to the ability of these peptides to prevent the release of pro-inflammatory molecules from vacuoles in the leukocytes migrating into this space. This likely occurs through IL-10, an Interleukin involved in the anti-inflammatory mechanism documented in this patent.

These peptides have the ability to control the deposition of fibrin in the extravascular and subintimal spaces. While this activity has never been identified, this activity is intuitive in that the production of harmful deposits also triggers the mechanism by which the body should remove them. While this activity is delayed as are most of the activities of these peptides, the initiation of a mechanism to remove fibrin from the extravascular space is stimulated by the release of these peptides. The deposition of fibrin beneath the intima of blood vessels in vascular disease and the deposition of fibrin into the extra-vascular space in many other diseases results in the progression and exacerbation of these diseases. Over the last several years a great deal of research has focused on the regulation of fibrin, as mounting data suggests this deposition is a major part of many chronic disease processes. The discovery of therapeutics with the ability to regulate fibrin is now the impetus for extensive research, but the ability to regulate fibrin deposition has been elusive. The need for removal of these fibrin deposits is demonstrated by the impairment of function caused by the physical barrier fibrinogen forms, and by the pro-inflammatory activity of fibrin in these spaces. In addition, the presence of fibrin in these spaces has now been shown to suppress the activity of some cells which are essential for healing. One example of this is the ability of extra-vascular fibrin to inactivate the regenerative activity of Swann Cells. Over the last several years researchers have been able to demonstrate the benefit of removal of extra-vascular fibrin in many of these disease processes. These studies demonstrate the proinflammatory activity of fibrin as well as the impairment of normal cellular/organ function in their presence. This impairment is a major component of the pathologic process of many diseases, including but not limited to Multiple Sclerosis, Rheumatoid Arthritis, peripheral nerve crush injury, Alzheimer's Disease, Macular Degeneration, and Atherosclerosis. In these studies the researchers recognize extra-vascular fibrin as an important target for new therapeutics. These peptides have the ability to regulate both the deposition and resorption of fibrin, and are therefore a new treatment option for a wide variety of diseases.

af-FA, af-FB, and imf-C3 therefore initiate a complex interaction between cytokines and immune system cells that allows the patient's immune system to recognize and respond to the source of most chronic disease by enhancing the ability of the patient's immune system to better recognize foreign proteins. At the same time, the antibody response involved in autoimmune disease is decreased through the increased surveillance and elimination of B cells producing auto-antibodies, and through the potent anti-inflammatory effect. The treatment of autoimmune disease through this treatment also increases the evidence that most chronic illnesses are related to dysfunction of the immune system.

Therapeutic Functions—Filtered Product Containing af-FA, af-FB and imf-C3

### Treatment of Infectious Diseases

Viruses, Chronic (HIV, HBV, HPV, HSV, etc)

All chronic viral conditions result from the body's inability to recognize and eliminate a foreign substance associated with the virus. This is likely at least in part through a desensitization of the immune system to the proteins of that virus. Through the mechanism of action of this therapeutic, the enhancement of the immune system allows for the recognition and elimination of any virus, eliminating the permissive tendency to tolerate the presence of viruses which are not causing acute symptoms. This enhanced ability to recognize, seek out and destroy virus allows for the detection and elimination of all types of viral infection, even encapsulated viruses.

### Acquired Immune Deficiency Syndrome (AIDS)

AIDS is a disease in which a virus HIV infects and destroys cells of the immune system and can be life threatening when a specific type of T-lymphocytes called CD4 lymphocytes level drops to below 200/mcl. At this level the body looses cellular (acquired) immunity. This places the host at risk for a variety of diseases which are normally prevented by this portion of the immune system. Patients suffering from this syndrome suffer the symptoms of the opportunistic diseases, but HIV infection is otherwise asymptomatic except a mild flu like illness shortly after initial infection. As the virus hides in immune and other cells, the body gradually adopts a permissive approach to the proteins manifest on the surface of these cells. When the virus begins to replicate more aggressively, the body fails to recognize and attack the abnormal proteins manifest on these cells, or even to attack the virus itself after it is released from these cells. The process of replication depends on reverse transcription of the viral RNA, so the western medicine approach centers on preventing this activity of the HIV viral RNA. While this approach has been successful at slowing disease progression, no medications have been found to date which destroy the virus and cure the disease.

The curative ability of these peptides when given to patients with HIV/AIDS has not been established. The mechanism of action of peptides of the invention does indicate several benefits to the HIV infected population, and the potential curative ability of these peptides. One of the prominent activities of these peptides is the conversion of the immune system from a permissive state back to an aggressive or active state. This allows the patient's own immune system to seek out and attack cells infected by this virus through T-Killer lymphocytes. In addition, the cytokine up-regulation stimulated by these peptides is expected to directly enhance the production of T-reg cells, including CD4 cells. These changes allow the immune system to seek out and destroy the virus and cells which have the virus hiding inside. The anti-inflammatory activity also diminishes the symptoms of opportunistic infections and augments the immunologic response to these infections.

### Acute (Influenza, Ponta Toro, HAV, etc.)

In addition to this benefit in chronic infection, this peptide greatly decreases the symptoms and severity of acute infection by decreasing the inflammatory and reactive response within tissues while enhancing the ability of the protective immune system to combat the virus. af-FA, af-FB and imf-C3 improve survival of animals in a variety of acute viral disease models.

### Bacteria

Bacterial infection represents a severe insult to the system. In the presence of this most emergent form of insult to the system af-FA, af-FB, and imf-C3 enhance the activities of the immune system which are most crucial to the elimination of the bacterial insult, while controlling the symptoms which result from increased inflammation. When a patient experiences a breach of the integument a significant exposure to bacteria occurs. Despite this exposure, patients rarely develop an infection at the site and even less frequently develop a systemic infection. At least part of this protective response occurs due to the release of Fibrinopeptides A and B into the bloodstream as fibrinogen is activated to seal the breech in the system. A similar benefit occurs when af-FA and af-FB are given to a patient that has been exposed to an acute infection. This effect is greater when the medication is administered prior to the exposure. However, whether administration is at the time of exposure or after the exposure, both are still beneficial in the process of eliminating the infection. This stimulation occurs through several different immune cells, including T cells, B cells and macrophages. T40 cells play a particularly important roll.

### Parasites

In the same way af-FA and af-FB and imf-C3 enhances the immune response to viruses and bacteria it enhances the ability of the body to respond to parasitic diseases whether chronic or acute.

Spirochetes are particularly difficult to treat, but with the immune system modulation produced by af-FA, af-FB and imf-C3 even these organisms are recognized and destroyed

Fungus and yeast infections are typically considered opportunistic, but with af-FA, af-FB, and imf-C3 the enhanced surveillance a patient experiences virtually eliminates the potential for this type of infection. For those suffering from this type of infection, the peptides enhance the ability of the immune system to respond and eradicate the infection.

### Treatment for Cancer

Cancer is a broad term describing a myriad of diseases with some common features: 1) Loss of cellular regulation; 2) abnormal replication; and 3) destruction of adjacent tissues through either infiltration or compression. These diseases can be caused by a variety of factors as well, including infection, radiation, exposure to toxins, and genetic predisposition. Once cells develop cancerous features, they are typically destroyed by the organism. When this induced apoptosis fails, cancer develops. The western medicine approach entails the use of radiation and chemotherapy to destroy cancerous cells, but these approaches are wrought with the difficulties of negative side effects.

Through enhanced surveillance and destruction of cells with abnormal proteins on their surface (a common feature of all cancer cells), these peptides have the ability to prevent and even treat cancer without all of the negative side effects of traditional western medicine cancer treatments. In addition to this direct benefit on the destruction of cancer, these peptides have the ability to ameliorate the symptoms of chemotherapy and radiation, as they decrease the inflammation the patients have in response to these therapeutic modalities.

Not only does this enhanced surveillance help the immune system recognize foreign bodies, it also enhances the process of apoptosis in eliminating any abnormal cells. Cancer cells are known to possess abnormal proteins on their surface. The failure of the immune system to recognize these proteins and trigger apoptosis in these cells allows cancer to progress. These peptides stimulate this process, allowing the patient's immune system to eliminate abnormal cells. Currently several different autologous vaccines are utilized to treat cancer. The current method theorizes that processing and then re-injecting cancer cells is responsible for the therapeutic benefit by allowing the body to recognize these abnormal proteins and then attack them. Given the method of processing of most of these vaccines, most probably still contain large quantities of the peptides of this invention and the exposure to the processed cell together with the ability of the immune system to more completely recognize these abnormal cells leads to the therapeutic benefit. The peptides of this invention in and of them selves have this same ability.

In addition to this benefit, these peptides decrease the leakage of blood vessels, and this benefit decreases the deposition of fibrin around tumors, making them more susceptible to the attack of the immune system.

IL-1b is preferably used as an adjunct to cancer treatment in an effort to minimize the insult to the immune system of some chemotherapeutic agents. As these peptides stimulate the release of this cytokine, the benefit in this therapeutic indication is obvious. In addition, the anti-inflammatory activity of these peptides greatly improves the tolerability of chemotherapy and radiation therapy, decreasing the pain and suffering associated with these treatments.

As the side effects are very minimal and the immune system is not suppressed, this option for treating cancers of all varieties holds many advantages over conventional treatment. In addition, the lack of destruction of other cells in the body eliminates the need to tolerate the very negative side effects of chemotherapy.

### Treatment for Coronary Artery Disease (CAD)

Coronary Artery Disease (and vascular disease of all types) develops when the intima of the blood vessel wall is injured, or when the lipids in the bloodstream are high enough that they begin to deposit in the subintimal space. Either injury or lipid deposition results in a progressive condition leading eventually to severe narrowing of the blood vessel. In these lesions, lipids represent approximately thirty percent of the lesion, while the other seventy percent contains the deposition of fibrin, iron, and other proteins. Traditional Western medicine utilizes therapeutics with a powerful lipid lowering potential, but these also carry considerable risk of side effects. Besides those with severe side effects, many patients taking these medications do not feel well and have muscle pain with minimal exercise or even at rest. This type of treatment has been shown to reduce the risk of Coronary Artery Disease events.

While controlling this deposition of lipids has always been and will continue to be an important aspect of CAD treatment, the ability of these peptides to mobilize the fibrin deposits in the wall of the blood vessel will have a greater effect on the long term health of patients suffering from this disease than simply trying to slow the deposition of lipids. By simple math it is easy to see the benefit of treating the problem occupying 70% of the lesion over addressing the problem occupying thirty percent. The lipid portion of the plaque is also protected by a fibrinous cap. These peptides enhance the ability of the body to take up this lipid portion of the plaque by triggering the resorption of this protective fibrinous cap. The ability of these peptides to stop inflammation and enhance the resorption of fibrin and iron from the subintimal and extravascular spaces has obvious therapeutic benefits for all vascular diseases.

### Treatment for Osteoarthritis

Many painful conditions are just the result of degeneration of normal tissues through the aging process. While this process in and of its self does not cause pain, these broken down tissues do stimulate the inflammatory cascade which result in chronic pain and stiffness. Osteoarthritis is an example of this type of chronic painful condition. The initial process seems to be overuse or injury, but the subsequent inflammation also contributes to the degenerative process. While these peptides cannot restore the degenerated tissue, they do stop the secondary inflammatory process. By blocking this process, the amount of pain and the speed of continued degeneration are both decreased dramatically.

### Treatment for Autoimmune Disease (Rheumatoid Arthritis, Lupus Erythematosus, Scleroderma, etc.)

Each of the auto-immune diseases has specific symptoms of only that disease. Auto-immune disease begins with a genetic component in which the HLA haplotype response to a pathogen results in the production of an antibody which attacks not only the pathogen, but also a constituent part of the patient's body. This happens in all individuals, but when the patient's immune system looses the ability to eliminate B-cells with this activity, the result is a persistent stimulation and perpetuation of these cells as the antibodies seek out and destroy the “pathogenic insult”, in this case the patient's own tissue. As these antibodies attach to the patient's tissues, they trigger an inflammatory cascade which results initially in tissue destruction, and then in fibrin and iron deposition which further perpetuate the inflammatory response. This creates a vicious cycle of destruction of tissue, pain, and progression of disease pathogenesis.

These peptides ameliorate the health of patients with autoimmune disease by: 1) enhancing the body's mechanism of seeking out and destroying cells which produce auto-antibodies, 2) decreasing the local inflammation responsible for many of the acute symptoms, 3) blocking the deposition and stimulating the resorption of harmful extravascular fibrin and iron deposits, a consequence of the bodies reaction to the auto-antibodies, and 4) stimulating the absorption of fibrin from the blood vessel intima which causes the venous insufficiency. These activities all result from the ability of these peptides to stimulate the release of IL-1B, IL-10, and IL-13 from macrophages and monocytes, and through the stimulation of removal of harmful extravascular substances.

### Treatment for Multiple Sclerosis

Multiple Sclerosis is a disease with many characteristics leading up to the progression of neurologic induced disability. In this disease, the initial event appears to be an autoimmune attack of the myelin in the central nervous system. As these areas of injury progress, acute inflammation around these areas of autoimmune attack causes varying degrees of neurologic improvement, which initially resolve as the inflammation diminishes. With each “MS attack”, the lesions progress, the nerve injury becomes more extensive, and the deposition of fibrin and iron into the space surrounding the lesion becomes more extensive. As these deposits advance, they create a more extensive inflammatory response, stop the ability of these areas to heal, and therefore cause the disease to further progress. This process also progresses along the venous drainage of the affected area, causing an increase in venous pressure. In addition, fibrin deposition in MS causes a fibrin cuff around blood vessels in many disease processes, including MS. This fibrin cuff slows blood flow, decreases perfusion of nutrients, and results in vascular congestion. The resulting venous insufficiency and its role in the progression of MS is now the source of considerable debate in the scientific literature and among neurologists. Placing stents in these veins at areas of occlusion is a treatment under investigation in progressive MS, with preliminary data from Europe and Canada showing substantial improvement in most patients. While the investigators claim this improvement proves that MS is a vascular disease rather than auto-immune, their data reinforces the multifactorial nature of this very complicated disease.

These peptides have the ability to treat multiple sclerosis through a reverse of this cascade. It does this by: 1) enhancing the body's mechanism of seeking out and destroying cells which produce auto-antibodies, 2) decreasing the local inflammation responsible for many of the neurologic symptoms, 3) blocking the deposition and stimulating the resorption of harmful extravascular fibrin and iron deposited due to the increase in vascular leakage of these substances into the extravascular space, and 4) stimulating the absorption of subintimal fibrin, the cause of the venous insufficiency in progressive MS. These activities all result from the ability of these peptides to stimulate the release of IL-1B, IL-10, and IL-13 from macrophages and monocytes, and through the stimulation of removal of harmful extravascular substances. Each of these activities is discussed at length in the background sections above.

### Treatment for Alzheimer's Disease

The cause of Alzheimer's Disease remains elusive, but once again appears to be a multifactorial problem. Current consensus attributes the onset to the deposition of β-amyloid protein in the connective tissue between neurons. This then stimulates the deposition of fibrin and iron, resulting in an effect somewhat like that described for MS, although predominantly in the memory centers as this seems to be the location most susceptible to the deposition of β-amyloid protein. Once this protein is deposited, the Blood-Brain Barrier (BBB) becomes leaky, allowing the body to encase the β-amyloid protein in a fibrin network and forming neuritic plaques. This suggests the body's recognition of this protein as a harmful substance in this location. These fibrin deposits then increase the localized inflammatory response, leading to the progression of the disease. Cortes-Cantoneli et. al. (2009) demonstrated this causative effect of fibrin deposition through a series of experiments designed to first increase fibrin deposition, and then to reduce fibrin deposition. Many other researchers have also demonstrated the role of inflammation in the development of Alzheimer's Disease. In addition, the vascular changes of Alzheimer's Disease either cause or are a result of these abnormal protein deposits.

While much of the expected benefit in Alzheimer's Disease is speculative, these peptides should have a tremendous effect by mobilizing the protein deposits, eliminating the inflammation, and enhancing/improving blood flow to and from the damaged tissues.

### Treatment for Chronic Wounds

af-FA, af-FB, and imf-C3 greatly enhance healing in chronic wounds, this occurs through a variety of activities and through the direct stimulation of fibroblasts by these peptides as well as the enhanced angiogenesis stimulated by the cytokine cascade. In addition, most chronic wounds have a chronic low grade infection. af-FA, af-FB, and imf-C3 stimulate the immune system to recognize and eliminate this chronic infection, speeding healing. Use as a treatment for hypersensitivity reactions

The ability of af-FA to deglycosolate and thereby inactivate IgE is established. In addition to this activity, these peptides trigger the release of IL-10 and IL-13. IL-10 undoubtedly plays a prominent role in this process. Even in using animal serum for the procurement of these peptides and then injecting fragments containing larger proteins, there does not seem to be any anaphylactic potential in these serum fractions.

### Treatment for Chronic Inflammatory Conditions

The profound anti-inflammatory action of af-FA, of-FB, and imf-C3 results in the amelioration of all types of chronic inflammation. Patients with inflammation secondary to infection, autoimmune disorders, and degenerative disease will experience a decrease in their pain symptoms with administration of these peptides.

### Treatment for Chronic Pain

Shortly after a significant injury occurs, patients report a period of relatively less pain. The response seen in patients to an injection of these peptides indicates that this cytokine expression modification also causes a change in nerve function, decreasing the sensitivity of pain fibers. The shift from a Th1 (pro-inflammatory) to a Th2 (anti-inflammatory) state also plays a significant role in the treatment of chronic pain.

### Treatment for Diabetic Ulcers

Diabetic wounds occur as a result of two different processes. The first is the development of chronic arterial insufficiency in the small arterioles and capillaries. The overlying tissue does not get sufficient blood flow to sustain life, and therefore breaks down and ulcerates. The bed is then open but, due to ongoing difficulty with poor circulation, the base of the wound bed still does not have sufficient blood flow to promote healing. The ulcer therefore becomes a chronic wound, and eventually will get infected and necessitate amputation. The other type of diabetic ulcer results from diabetic neuropathy. In this type of wound the patient does not have sufficient feeling in the affected body part to recognize a consistent inappropriate source of pressure (e.g.—poorly fitting shoes or a foreign body in the shoe). This creates a pressure ulcer, but the lack of proper innervation and the poor circulation both prevent proper healing.

The peptides found in these serum fractions help in this healing process through several effects: 1) they open up the blood vessels through the effect on both lipid and fibin deposition that is causing the poor blood flow; 2) they decrease the inflammatory changes around the nerves and promote the remyelinization of nerve cells; 3) they break down the fibrinous layer which forms at the base of these wounds; 4) they promote an anti-inflammatory environment which promotes healing; 5) they stimulate the immune system to address the infectious component of these chronic wounds; 6) they stimulate the replication and migration of fibroblasts; 7) they stimulate the differentiation of vascular cells promoting angiogenesis; and 8) they promote the migration of macrophages into the wound bed to enhance elimination of any substances that may slow healing. These effects transform the site of the wound from one that suppresses the body's ability to heal to an environment that promotes rapid healing.

### Reflex Sympathetic Dystrophy

Reflex Sympathetic Dystrophy (RSD), also called Complex Regional Pain Syndrome (CRPS), is a neurologic disorder of the Peripheral Nervous System. The key symptom of RSD is continuous, intense pain out of proportion to the severity of the injury, which gets worse rather than better over time. RSD most often affects one of the arms, legs, hands, or feet. Often the pain spreads to include the entire arm or leg. Typical features include dramatic changes in the color and temperature of the skin over the affected limb or body part, accompanied by intense burning pain, skin sensitivity, sweating, and swelling. The cause of RSD remains unclear. In some cases the sympathetic nervous system plays an important role in sustaining the pain. Another theory suggests RSD is caused by a triggering of the immune response, which leads to the characteristic inflammatory symptoms of redness, warmth, and swelling in the affected area. Because there is no cure for RSD, current accepted treatment is aimed at just relieving painful symptoms.

While the cause of RSD is unclear, the benefit of these peptides is established. They decrease pain experienced by RSD patients due to improvements in both the immune system and a decrease in the inflammation of nerve cells. After taking these peptides, patients experience an almost immediate relief of many of their painful symptoms, a benefit which persists over time. The response seen in patients to an injection of these peptides indicates that this cytokine expression modification also causes a change in nerve function, decreasing the sensitivity of pain fibers. The shift from a Th1 (pro-inflammatory) to a Th2 (anti-inflammatory) state likely also plays a significant role in the treatment of chronic pain.

### Use as a Treatment for Neurologic Disorders Including Seizures, Parkinson's Disease, and Even Schizophrenia.

af-FA, of-FB and imf-C3 decrease the activity in stimulated nerve cells and decrease the inflammation around these cells. This response not only has a profound effect on pain nerves, but it also plays an important role in the treatment of seizure, Parkinson's disease, Multiple Sclerosis, and even schizophrenia.

### Prevention of Disease

This brief summary discusses only a few of the many diseases in which these peptides have tremendous therapeutic benefit. However, perhaps the greatest use of the peptides is in the prevention of disease. Taken regularly, the activity of these peptides has essentially an anti-aging benefit. When taken as a whole, the benefits of taking these peptides diametrically oppose the established aging process. This is perhaps best illustrated in the ability of Peptide A to block the damage to cells and tissues by thermal and chemical burns. After administration of one of these peptides subcutaneously (Peptide A), the amount of chemical or thermal insult required to attain the same degree of burn increased substantially. This demonstrates the protective effect of these peptides. Furthermore, the role of fibrin deposition in the aging process is well established. The ability to prevent these deposits will therefore greatly slow the aging process. The immune response is also changed to be more like the immune response of a child, without losing the acquired immunity present in adults. This change in the functioning of the immune system prevents these changes of aging and even reverses the existing aging process to some degree.

### Examples of Implementation of the Invention

Natural Serum Fractions containing af-FA, af-FB and imf-C3: Many serum fractions containing these peptides are already being produced and test for the treatment of disease. However, these peptides have not been recognized as the active component of these preparations.

Synthetic af-FA, af-FB, and imf-C3: The natural forms of these peptides have not been previously identified in any established therapeutic, but may be the active ingredient in some therapeutics. A synthetic form of Fibrinopeptide A in humans is also readily available for laboratory use, but a form acceptable for animal or human use is not readily available. In addition, the removal of the terminal Arginine to activate the molecule has not been identified and is only available through custom synthesis. As this activation is important for the therapeutic effect, and as the carboxypeptidase B activity is limited in humans to a greater degree than in other mammals, a custom synthesis is preferred.

To establish the therapeutic activity and compare unactivated (with terminal Arginine still attached) af-FA, af-FB, and imf-C3 with the active form, comparison testing using the naturally obtained peptide as well as synthetic forms of the peptide must be performed. From preliminary data, the synthetic form of af-FA, af-FB, and imf-C3 is comparable to the natural form as far as bioactivity. In addition, the activated form (terminal Arginine removed) is much more bioactive than the unactivated form. This data strongly supports the benefit of synthetic af-FA, af-FB, and imf-C3 as a therapeutic for many disease processes including the above delineated processes.

Synthetic product with similar characteristic: In addition to using the exact sequence, the structural homology of this peptide from different species indicates that any peptide with similar structure will be biologically active. While any form of peptide with these structural similarities is likely to contain the same biological activity, the naturally derived sequences are expected to be the safest and most active.

Autologous vaccine: In addition to the therapeutic activity of the natural and synthetic forms of both animal and human af-FA af-FB and imf-C3, this data indicates a patient's own blood can be utilized as the source of obtaining these peptides. The patient's blood is obtained in a manner similar to a routine blood draw for analysis, run through a simple process to encourage the release and activation of the patient's own af-FA and af-FB, and then these peptides are filtered and reinjected into the patient. This process eliminates all of the complications associated with a foreign protein of any type, and can be utilized to treat many different disease processes, including those above.

Immunization for prevention of disease: The mechanism of action of af-FA, af-FB, and imf-C3 establishes the potential for the utilization of these molecules as a method to prevent disease. In patients with a known exposure to a pathogen, this therapeutic enhances the body's ability to eliminate the illness before the organism becomes symptomatic.

Utilization as a vaccine adjunct: The enhanced B cell life and increased activity toward foreign molecules also indicates the potential for this therapeutic to be utilized as an adjunct to current vaccinations, and allows vaccine molecules to be presented in an environment that augments the organism's response.

TABLE 7
Sequence ID information

SEQ ID NO. 1: ADSGEGDFLAEGGGVR (Human Fibrinopeptide A).
SEQ ID NO. 2: ADSGEGDFLAEGGGV (Human Fibrinopeptide A activated by
removal of the terminal Arginine).
SEQ ID NO. 3: EDGSDPPSGDFLTEGGGVR (Bovine sequence analogous to
human Fibrinopeptide A in location and sequencing in the Fibrinogen
Alpha Chain, also a portion of the AAI02565 protein).
SEQ ID NO. 4: EDGSDPPSGDFLTEGGGV (Bovine sequence Fibrinogen Alpha
Chain activated by removal of the terminal Arginine).
SEQ ID NO. 5: EDGSDPPSGDFLTEGGGV with hydration or other modification.
SEQ ID NO. 6: TDYDEGQDDRPKVGLGA with a sulfate attached
(a portion of the Fibrinogen Beta Chain Sequence).
SEQ ID NO. 7: SEETKENERFTV (portion of bovine protein AAI12453 from
Complement C3 protein and from caprine Complement C3 protein).
SEQ ID NO. 8: TEEGEFLHEGGGVR (homologous sequence of equine
fibrinogen alpha chain to Fibrinopeptide A).
SEQ ID NO. 9: TEEGEFLHEGGGV (homologous sequence of equine fibrinogen
alpha activated by removal of the terminal Arginine).
SEQ ID NO. 10: DHEEEDGRTKVTFDA (Portion of the equine fibrinogen beta
chain).
SEQ ID NO. 11: ADDSDPVGGEFLAEGGGVR (Caprine Fibrinopeptide A).
SEQ ID NO. 12: ADDSDPVGGEFLAEGGGV (Caprine Fibrinopeptide A
activated by removal of the terminal Arginine).
SEQ ID NO. 13: DDSDPVGGEFLAEGGG (Caprine Fibrinopeptide A degradation
product much more prominent than any other degradation product
besides SEQ ID NO 12).
SEQ ID NO. 14: GYLDYDEVDDNRAKLPLDA with a sulfate group attached to
tyrosine (portion of the Caprine Beta Chain).
SEQ ID NO. 15: SEETKENEGFTV (human homologous sequence to Complement
C3 protein identified in both the caprine and the bovine samples)
SEQ ID NO. 16: SEETKENEGFTVTAEGK (sequence cleaved in vivo in human
specimens)
SEQ ID NO. 17: GVNDNEEGFFSAR (human Fibrinopeptide B)
SEQ ID NO. 18: GVNDNEEGFFSA (human Fibrinopeptide B activated by
the removal of the terminal Arginine)
SEQ ID NO. 19: NDNEEGFFSA (Active fragment of Fibrinopeptide B
found in human serum samples)
SEQ ID NO. 20: SEETKENE . . . FLAEGGGV (Spliced product of Amine
terminus of SEQ ID NO. 15 and SEQ ID NO. 21)
SEQ ID NO. 21: GGV (the minimum sequence necessary to produce the
activity of Fibrinopeptide A as demonstrated herein)
SEQ ID NO 22: FLAEGGGV (interspecies conserved region)

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. Furthermore, the term “comprising” includes the terms “consisting of and “consisting essentially of.” It is intended that the specification and examples be considered exemplary only with the true scope and spirit of the invention indicated by the following claims.

1. An agent comprising a peptide containing a sequence selected from the group consisting of SEQ ID NOs. 1-5, 7-9, 11-13, 15, 16, and 20-22, a sequence of Fibrinopeptide A, a sequence of a region of Fibrinopeptide A that is substantially homologous between species of mammals that produce fibrinopeptide A, a sequence of Compliment C3, and a foregoing sequence containing one or more conservative amino acid substitutions, wherein the agent contains substantially no detectable Fibrinopeptide B.

2. The agent of claim 1, further comprising a pharmaceutically acceptable carrier.

3. The agent of claim 2, wherein the pharmaceutically acceptable carrier is selected from the group consisting of water, oil, edible oil, fatty acids, lipids, polysaccharides, cellulose, glycerin, glycol, and combinations thereof.

4. The agent of claim 3, wherein the edible oil is lemon oil, peppermint oil, or grape seed oil.

5. The agent of claim 1, which is formulated for oral, transmucosal, parenteral, lymphatic, or intravenous administration.

6. The agent of claim 1, wherein a biologically active form of the agent is released into a system of a patient at a physiologically effective concentration.

7. The agent of claim 1, which is a dietary supplement.

8. The agent of claim 1, which is purified from a biological source or synthetically manufactured.

9. A pharmaceutical composition comprising Fibrinopeptide A or a fragment thereof, and a pharmaceutically acceptable carrier, wherein the Fibrinopeptide A or fragment thereof is at a therapeutically effective amount and the therapeutically effective amount is from 0.1 mg to 500 mg.

10. The composition of claim 9, wherein the therapeutically effective concentration prevents deposition and stimulates resorption of fibrin within the extravascular and subintimal spaces of a patient.

11. The composition of claim 9, wherein the therapeutically effective concentration prevents deposition and stimulates resorption of fibrin deposits associated with coronary artery disease.

12. The composition of claim 9, which is nontoxic at the therapeutically effective concentration and substantially free of detectable Fibrinopeptide B.

13. The composition of claim 9, wherein the Fibrinopeptide A or fragment thereof is derived from a human sequence of Fibrinopeptide A.

14. The composition of claim 9, wherein the Fibrinopeptide A or fragment thereof is derived from a nonhuman sequence of Fibrinopeptide A.

15. The composition of claim 14, wherein the non-human sequence is derived from a mammal.

16. The composition of claim 15, wherein the mammal is selected from the group consisting of an equine, a feline, a canine, a bovine, a caprine, an ovine, and a murine.

17. A method for treating or preventing a disorder of a patient comprising:
providing a pharmaceutical composition comprising Fibrinopeptide A or a fragment thereof and not Fibrinopeptide B, and a pharmaceutically acceptable carrier, wherein the Fibrinopeptide A or fragment thereof, is derived from a mammal that is not a human; and
administering a dose of the composition to the patient, wherein administration is transmucosal such that the Fibrinopeptide A or fragment thereof achieves a therapeutically effective level within the lymphatic system of the patient within 5 minutes of administration.

18. The method of claim 17, wherein the patient is a human.

19. The method of claim 17, wherein the disorder is vascular inflammation.

20. The method of claim 17, wherein the disorder is coronary artery disease.

21. The method of claim 17, wherein a single dose contains from 0.1 mg to 10 mg of active ingredient.

22. The method of claim 17, wherein administering the dose of the composition to the patient comprises an initial administration and subsequently a continued administration, and the continued administration is not repeated for at least 7 days.

23. The method of claim 17, wherein the transmucosal administration is oral.

24. The method of claim 17, wherein the Fibrinopeptide A or fragment thereof stimulates the release of cytokines IL1β, IL-10, and not IL-1, IL-4 or TNF\dot{α}.

25. The method of claim 17, wherein activity of Fibrinopeptide B of the patient is suppressed.

26. The method of claim 25, wherein the activity of Fibrinopeptide B is suppressed by the administration of a Fibrinopeptide B binding agent.

27. A method of preventing, deposition of fibrin and absorbing Fibrin deposited within blood vessels of a patient, comprising:
providing a pharmaceutical composition that comprises Fibrinopeptide A or a fragment thereof and a pharmaceutically acceptable carrier: and
administering the composition to a patient such that the Fibrinopeptide A or fragment thereof is at a therapeutically effective level is achieved in the lymphatic system of the patient.

28. The method of claim 27, wherein the patient is human.

29. The method of claim 27, wherein the Fibrinopeptide A or fragment thereof is derived from a mammalian sequence of Fibrinopeptide A that is not a human.

30. The method of claim 27, wherein the composition is administered directly to the lymphatic system by transmucosal administration.

31. The method of claim 27, wherein administration to the patient comprises an initial administration and subsequently a continued administration, and the continued administration is no more than once a week.

32. A fraction of serum of a mammal wherein the fraction contains multiple components, is clarified of particulates, and substantially all components are within a molecular weight range of from about 1,200 Daltons to about 1,700 Daltons.

33. The fraction of claim 32, wherein the mammal is selected from the group consisting of an equine, a feline, a canine, a bovine, a caprine, an ovine, and a murine.

34. An agent comprising a peptide containing a sequence selected from the group consisting of SEQ ID NOs. 6, 10, 14, and 17-19, a sequence of Fibrinopeptide B, and a sequence of a region of Fibrinopeptide B that is substantially homologous between species of mammals that produce Fibrinopeptide B, wherein the agent contains substantially no detectable Fibrinopeptide A.

35. The agent of claim 34, further comprising a pharmaceutically acceptable carrier.

36. The agent of claim 35, wherein the pharmaceutically acceptable carrier is selected from the group consisting of water, oil, edible oil, fatty acids, lipids, polysaccharides, cellulose, glycerin, glycol, and combinations thereof.

37. The agent of claim 36, wherein the edible oil is lemon oil, peppermint oil, or grape seed oil.

38. The agent of claim 34, which is formulated for oral, transmucosal, parenteral, lymphatic, or intravenous administration.

39. The agent of claim 34, wherein a biologically active form of the agent is released into a system of a patient at a physiologically effective concentration.

40. The agent of claim 34, which is a dietary supplement.

41. The agent of claim 34, which is purified from a biological source or synthetically manufactured.

42. A method for treating or preventing a disorder of a patient comprising:
providing a pharmaceutical composition comprising Fibrinopeptide B or a fragment thereof, wherein the composition contains substantially no detectable Fibrinopeptide A, and a pharmaceutically acceptable carrier, wherein the Fibrinopeptide B or fragment thereof, is derived from a mammal that is not a human; and
administering a dose of the composition to the patient, wherein administration is transmucosal such that the Fibrinopeptide B or fragment thereof achieves a therapeutically effective level within the lymphatic system of the patient within 5 minutes of administration.

43. The method of claim 42, wherein the patient is a human.

44. The method of claim 42, wherein the disorder is an auto-immune disorder,

45. The method of claim 44, wherein the auto immune disorder is selected from the group consisting of arthritis, Crohn's disease, Coeliac disease, diabetes mellitus type I, Grave's disease, idiopathic thrombocytopenic purpura, psoriasis, scleroderma, systemic lupus erythematosus, and ulcerative colitis.

46. The method of claim 42, wherein the disorder is a immunoregulatory disorder.

47. The method of claim 46, wherein the immunoregulatory disorder is an overactive immune system.

48. The method of claim 42, wherein a single dose contains from 0.1 mg to 10 mg of active ingredient.

49. A fraction of serum of a mammal wherein the fraction contains multiple components, is clarified of particulates, and substantially all components are within a molecular weight range of from about 800 Daltons to about 2,300 Daltons.

50. The fraction of claim 49, wherein the mammal is selected from the group consisting of an equine, a canine, a feline, a bovine, a caprine, an ovine, and a murine.


SEQUENCE LISTING (22 sequences)

SEQ ID NO: 1 - protein, 16 aa, Homo sapiens
ADSGEGDFLAEGGGVR

SEQ ID NO: 2 - protein, 15 aa, Homo sapiens
ADSGEGDFLAEGGGV

SEQ ID NO: 3 - protein, 19 aa, Bos sp.
EDGSDPPSGDFLTEGGGVR

SEQ ID NO: 4 - protein, 18 aa, Bos sp.
EDGSDPPSGDFLTEGGGV

SEQ ID NO: 5 - protein, 18 aa, Artificial Sequence
  Description of Artificial Sequence Synthetic peptide
EDGSDPPSGDFLTEGGGV

SEQ ID NO: 6 - protein, 17 aa, Artificial Sequence
  Description of Artificial Sequence Synthetic peptide
TDYDEGQDDRPKVGLGA

SEQ ID NO: 7 - protein, 12 aa, Bos sp.
SEETKENERFTV

SEQ ID NO: 8 - protein, 14 aa, Unknown
  Description of Unknown Equine fibrinogen alpha sequence
TEEGEFLHEGGGVR

SEQ ID NO: 9 - protein, 13 aa, Unknown
  Description of Unknown Equine fibrinogen alpha sequence
TEEGEFLHEGGGV

SEQ ID NO: 10 - protein, 15 aa, Unknown
  Description of Unknown Equine fibrinogen beta sequence
DHEEEDGRTKVTFDA

SEQ ID NO: 11 - protein, 19 aa, Unknown
  Description of Unknown Caprine fibrinopeptide A sequence
ADDSDPVGGEFLAEGGGVR

SEQ ID NO: 12 - protein, 18 aa, Unknown
  Description of Unknown Caprine fibrinopeptide A sequence
ADDSDPVGGEFLAEGGGV

SEQ ID NO: 13 - protein, 16 aa, Unknown
  Description of Unknown Caprine fibrinopeptide A sequence
DDSDPVGGEFLAEGGG

SEQ ID NO: 14 - protein, 19 aa, Artificial Sequence
  Description of Artificial Sequence Synthetic peptide
GYLDYDEVDDNRAKLPLDA

SEQ ID NO: 15 - protein, 12 aa, Homo sapiens
SEETKENEGFTV

SEQ ID NO: 16 - protein, 17 aa, Homo sapiens
SEETKENEGFTVTAEGK

SEQ ID NO: 17 - protein, 13 aa, Homo sapiens
GVNDNEEGFFSAR

SEQ ID NO: 18 - protein, 12 aa, Homo sapiens
GVNDNEEGFFSA

SEQ ID NO: 19 - protein, 10 aa, Homo sapiens
NDNEEGFFSA

SEQ ID NO: 20 - protein, 16 aa, Artificial Sequence
  Description of Artificial Sequence Synthetic peptide
SEETKENEFLAEGGGV

SEQ ID NO: 21 - protein, 3 aa, Unknown
  Description of Unknown Fibrinopeptide A sequence
GGV

SEQ ID NO: 22 - protein, 8 aa, Unknown
  Description of Unknown Interspecies conserved sequence
FLAEGGGV